(19) United States
(12) Patent Application Publication
(10) Pub. No.: US 2010/0196887 A1
(43) Pub. Date: Aug. 5, 2010

(54) COMPOSITIONS AND METHODS FOR MULTIPLEX ANALYSIS OF POLYNUCLEOTIDES

(75) Inventors: Jens J. Hyldig-Nielsen, Moss Beach, CA (US); Mark J. Fiandaca, Princeton, MA (US); James M. Coull, Westford, MA (US)

Correspondence Address: LIFE TECHNOLOGIES CORPORATION;C/O INTELLEVATE, P.O. BOX 52050, MINNEAPOLIS, MN, 55402, US

(73) Assignee: APPLIED BIOSYSTEMS, LLC, Carlsbad, CA (US)

(21) Appl. No.: 12/480,624

(22) Filed: Jun. 8, 2009

Related U.S. Application Data
(63) Continuation of application No. 10/782,646, filed on Feb. 18, 2004, now abandoned.
(60) Provisional application No. 60/448,440, filed on Feb. 18, 2003.
(60) Provisional application No. 60/453,791, filed on Mar. 10, 2003.

(51) Int. Cl. C12Q 1/68

(52) U.S. Cl. 435/6

(57) ABSTRACT

Provided herein are compositions and methods for the multiplex analysis and/or detection of polynucleotides having one or more distinguishable target sequences. The methods employ signal-quencher probe pairs having specific relative differential thermal melting temperatures that permit the detection of one or more target sequences on one or more polynucleotides.

### CROSS-REFERENCES TO RELATED APPLICATIONS

This application claims benefit under 35 U.S.C. §119(e) to application Ser. No. 60/448,440, entitled “Compositions and Methods for Multiplex Analysis of Polynucleotides,” filed Feb. 18, 2003 and to application Ser. No. 60/453,791, entitled “Compositions and Methods for Multiplex Analysis of Polynucleotides,” filed Mar. 10, 2003, the disclosures of which are incorporated herein by reference in their entirety.

### FIELD

The present disclosure relates to compositions and methods for the multiplex analysis of polynucleotides using probe pairs having specified relative thermal melting temperatures.

### BACKGROUND

Nucleic acid hybridization is a fundamental phenomenon in molecular biology. Probe-based assays that exploit sequence-specific hybridization are used in many applications for detecting, analyzing and quantifying nucleic acids. For example, probe-based hybridization is at the core of numerous assays commonly employed to quantify gene expression levels, to detect single nucleotide polymorphisms (SNP) and other genetic mutations, as well as to type, map and/or fingerprint genes.

Oftentimes, such assays are carried out in a multiplex fashion with probes bearing different, distinguishable labels, permitting a multiplicity of results to be obtained in a single assay reaction. For example, a polynucleotide sample may be assessed for the presence or absence of two or more different sequences of interest in a single assay using a plurality of different sequence-specific probes, each of which bears a different, distinguishable label, such as a fluorophore capable of emitting light at a unique, spectrally resolvable wavelength. In such an assay, the polynucleotide sample is contacted with the plurality of labeled sequence-specific probes under conditions in which the probes hybridize to their respective complementary sequences, if present. Following washing to remove unhybridized probes, the assay reaction is assessed for the presence or absence of specific spectral signals or colors, the presence of which correlate with the presence of particular sequences of interest.

While such multiplex assays are powerful, the number of different sequences that can be assessed in a single assay reaction is limited by several factors, including, for example, the number of different, distinguishable labels available and the availability of detection equipment capable of detecting the signals produced by the different, distinguishable labels. The degree of complexity of such multiplex assays would be greatly increased by the ability to distinguish hybridization events involving different sequence-specific probes bearing either common or indistinguishable labels. Accordingly, there is a need for multiplex polynucleotide assays that are not limited by the availability of different, distinguishable labels or equipment capable of detecting such labels.

### SUMMARY

The present disclosure provides compositions and methods for the multiplex analysis of polynucleotide samples. The compositions and methods described herein employ sequence-specific signal-quencher probe pairs having differential relative thermal melting temperatures (Tₘ) that permit the detection of one or a plurality of target sequences in a single assay without having to use different, distinguishable labels. Indeed, by virtue of the use of quencher probes having specified relative Tₘs, a plurality of different target sequences may be assessed in a multiplex fashion even in instances where all of the signal probes bear the same label. The use of signal probes bearing different, distinguishable labels increases the number of different target sequences that may be analyzed or detected in a single, multiplex assay. Thus, the compositions and methods described herein permit the multiplex analysis of polynucleotide samples by Tₘ, or by a combination of both Tₘ and label signal.

In some embodiments, the disclosure provides methods for contacting a polynucleotide sample suspected of containing one or more target sequences with at least two different signal-quencher probe pairs. The signal-quencher probe pairs can be designed to hybridize to target sequences located on one or more polynucleotides. The sequences of the first signal-quencher probe pair can be designed so that they hybridize within quenching proximity to one another within a region of a first specified target sequence. The sequences of the second signal-quencher probe pair can be designed so that they hybridize within quenching proximity to one another within a region of a second specified target sequence. Each signal probe can bear a label capable of producing a detectable signal when the signal probe is hybridized to a target sequence. The signal produced by the first signal probe may be distinguishable from that produced by the second signal probe, or it may be indistinguishable from that produced by the second signal probe. Each quencher probe can bear a moiety capable of quenching the signal produced by its corresponding signal probe when the signal and quencher probes are hybridized within quenching proximity to one another on a target sequence.

Each signal-quencher probe pair can be designed so that the quencher probe has a lower Tₘ than its corresponding signal probe. In embodiments in which the signal probes bear indistinguishable labels, the second signal probe can be designed to have a lower Tₘ than the first quencher probe. In embodiments in which the signal probes bear distinguishable labels, the second signal probe can be designed to have a lower, higher or equivalent Tₘ than the first signal or first quencher probe.

Following contact, the signals produced by the signal probes can be monitored as a function of temperature. Such signals can be monitored continuously or at a plurality of different discrete points as the temperature is increased or decreased through a temperature range including the Tₘs of the various different probes. In some embodiments signals are monitored at a plurality of different discrete temperatures. For example, in some embodiments, temperatures that are halfway between the Tₘs of the signal and quencher probes of a signal-quencher probe pair, and halfway between the Tₘ of the of the quencher probe of the first pair and the Tₘ of the signal probe of the second pair, and so forth, may be used. In some embodiments, temperatures that are approximately equal to the Tₘs of the various signal and quencher probes can be used. The signals produced by the signal probes as a function of temperature provide an indication of whether the polynucleotide sample includes one or more target sequences.

The number of signal-quencher probe pairs employed in the methods can depend upon the number of target sequences to be assessed in the assay. For example, in diagnostic contexts, it is often desirable to determine not only whether a patient is, for example, infected with a virus, but also the specific genotype of the infecting virus. In this context, the multiplex assay may employ as many signal-quencher probe pairs as is required to determine the known genotypes of the virus. In some embodiments, the signal and quencher probes of each signal-quencher probe pair can be designed to hybridize to a sequence that is indicative of a specific genotype. In some embodiments, each signal probe can be designed to hybridize to a sequence that is indicative of a specific genotype and one or more of the quencher probes may be designed to hybridize to sequences that are common to the different genotypes. In some embodiments, each quencher probe can be designed to hybridize to a sequence that is indicative of a specific genotype. The signal probes may all bear indistinguishable labels or, alternatively, some or all of the signal probes may bear distinguishable labels.

In some embodiments, the quencher probe of the signal-quencher probe pair having the lowest Tₘ may optionally be absent.

Also provided are compositions and kits useful for carrying out the various methods described herein. Generally, the kits can comprise a first signal-quencher probe pair and at least a second signal quencher probe pair, where the quencher probe of the signal-quencher probe pair having the lowest Tₘ is optional. In some embodiments, the kits can comprise from 2 to 10 different signal-quencher probe pairs. In some embodiments, all of the signal probes can bear indistinguishable labels. In some embodiments, at least one signal probe can bear a distinguishable label. In some embodiments, the kits can comprise a first set of from 2 to 10 different signal-quencher probe pairs, all of which are labeled with a first distinguishable signal label and a second set of from 2 to 10 different signal-quencher probe pairs, all of which are labeled with a second distinguishable signal label, distinguishable from the first signal label. The kit may optionally include additional sets of from 2 to 10 different probe pairs, wherein all probe pairs of the additional sets can be labeled with signal labels distinguishable from the signal labels of all other sets.

By virtue of utilizing differential Tₘs, multiple target sequences can be analyzed simultaneously without the requirement of distinguishable labeling systems. Moreover, the use of quencher probes can be used to decrease signals from signal probe-target hybrids that are not perfectly complementary. The quencher probe can quench the signal from each non-complementary signal-target hybrid, effectively increasing the specificity of the signal probes. In addition, embodiments employing a combination of differential Tₘs and different, detectable signals (e.g., differently colored fluorophores), permits the investigation and/or analysis of a large number of different target sequences in a single assay. The number of target sequences that can be investigated or analyzed simultaneously is the product of the number of distinguishable detectable signals and number of distinguishable Tₘs; the only limit is the ability to differentiate Tₘs and detectable signals.

The compositions and methods described herein can find use in many applications for analyzing polynucleotide samples. As specific non-limiting examples, the compositions and methods may be used to analyze multiple mutations simultaneously, to detect polymorphisms, to detect the presence or absence of one or a plurality of infectious agents in a sample, and to genotype infectious agents, such as viruses. Many other uses and advantages will become apparent upon review of the detailed description of the preferred embodiments.

### BRIEF DESCRIPTION OF THE DRAWINGS

FIG. 1A illustrates an example of a signal-quencher probe pair in which both the signal probe and quencher probe are designed to hybridize within quenching proximity to a region of a target sequence comprising a “discriminating” nucleobase sequence that may be used to discriminate the target sequence from other target sequences;

FIG. 1B illustrates an example of a signal-quencher probe pair in which the signal probe is designed to hybridize to the region of the target sequence comprising a “discriminating” nucleobase sequence that can be used to discriminate the target sequence from other target sequences that may be present in a sample and the quencher probe is designed to hybridize to a region of the target sequence comprising a “non-discriminating” nucleobase sequence;

FIG. 1C illustrates an example of a signal-quencher probe pair in which the quencher probe is designed to hybridize to the region of the target sequence comprising a “discriminating” nucleobase sequence and the signal probe is designed to hybridize to a region of the target sequence comprising a “non-discriminating” nucleobase sequence;

FIG. 2A illustrates the basic principles of Tₘ multiplexing with reference to an example in which three different self-indicating signal probes bear indistinguishable signal labels and hybridize to the discriminating region of a target sequence;

FIG. 2B provides a theoretical signal profile obtained from the example of FIG. 2A as a function of decreasing temperature;

FIG. 2C provides a theoretical first derivative profile of the signal profile of FIG. 2B;

FIG. 2D provides a theoretical signal profile obtained from the example of FIG. 2A as a function of increasing temperature;

FIG. 2E provides a theoretical first derivative profile of the signal profile of FIG. 2D;

FIG. 3 illustrates one of the advantageous features of Tₘ multiplexing with signal-quencher probe pairs;

FIG. 4 illustrates an example that uses two-fold Tₘ and color multiplexing (i.e., a first set of signal-quencher probe pairs labeled with a first fluorescent signal label and a second set of signal-quencher probe pairs labeled with a second fluorescent signal label of a different color);

FIG. 5A provides an actual signal profile of an assay obtained using a Tₘ multiplexing method described herein;

FIG. 5B provides a first derivative of the signal profile of FIG. 5A;

FIG. 6A illustrates an example where the signal-quencher probe pair hybridizes to a target sequence present on the same strand of a polynucleotide;

FIG. 6B illustrates an example where the signal-quencher probe pair hybridizes to a target sequence present on different strands of a polynucleotide;

FIG. 7 provides a first derivative of the signal profile from the hybridization experiment described in Example 3.

### DETAILED DESCRIPTION

### Abbreviations and Conventions

The abbreviations used throughout the specification and in the FIGS. to refer to target sequences, polynucleotides, signal probes and quencher probes comprising specific nucleobase sequences are the conventional one-letter abbreviations. Capital letters represent nucleotide sequences (e.g., RNA and DNA sequences) and lower case letters represent nucleotide mimic sequences (e.g., PNA sequences). Thus, when included in a poly or oligonucleotide, the naturally occurring encoding nucleobases are abbreviated as follows: adenine (A), guanine (G), cytosine (C), thymine (T) and uracil (U). When included in a poly or oligonucleotide mimic, such as a PNA, the naturally occurring encoding nucleobases are abbreviated as follows: adenine (a), guanine (g), cytosine (c), thymine (t) and uracil (u). “Nucleobase sequence” or “sequence” are used interchangeably.

Also, unless specified otherwise; poly or oligonucleotide sequences that are represented as a series of one-letter abbreviations are presented in the 5′→3′ direction, in accordance with common convention. Poly or oligonucleotide mimic sequences that have amino and carboxy termini, such as PNAs, are presented in the amino-to-carboxy direction, in accordance with common convention. For the purposes of distinguishing parallel from anti-parallel hybridization orientation, it is understood that the 5′ terminus of an oligonucleotide corresponds to the amino terminus of a PNA and the 3′ terminus of an oligonucleotide corresponds to the carboxy terminus of a PNA.

### DEFINITIONS

As used throughout the specification and claims, the following terms are intended to have the definitions delineated below. Terms defined in the singular also include the plural and vice versa.

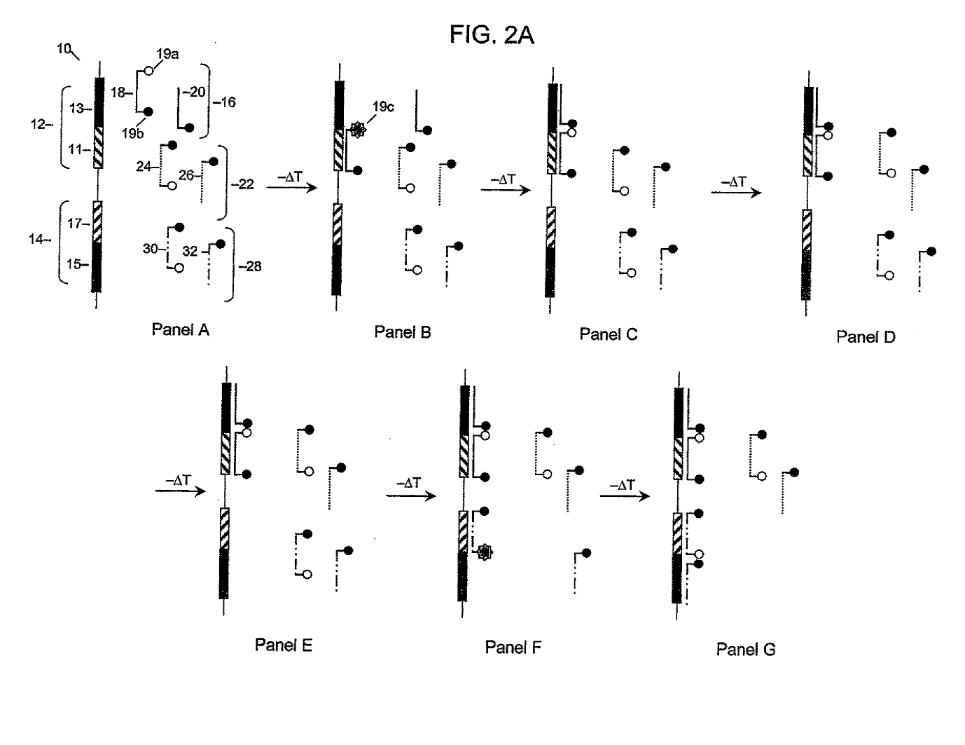

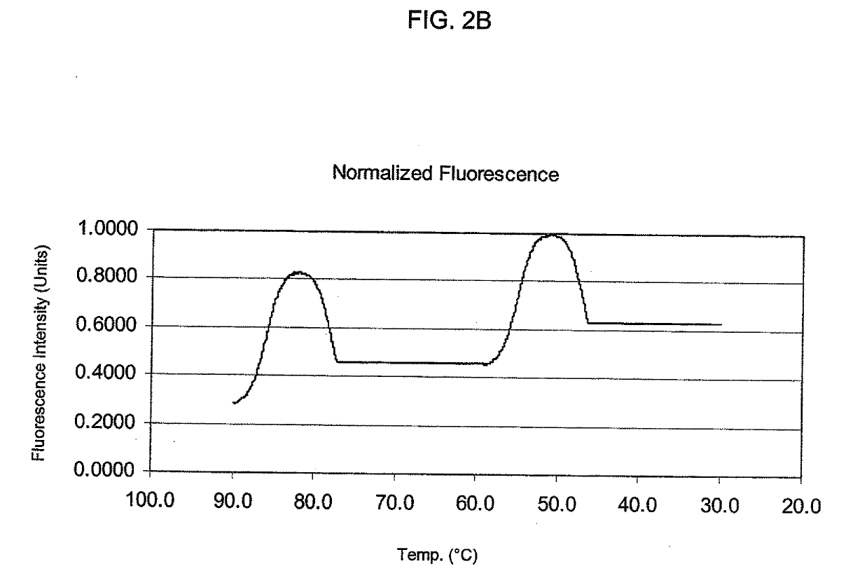

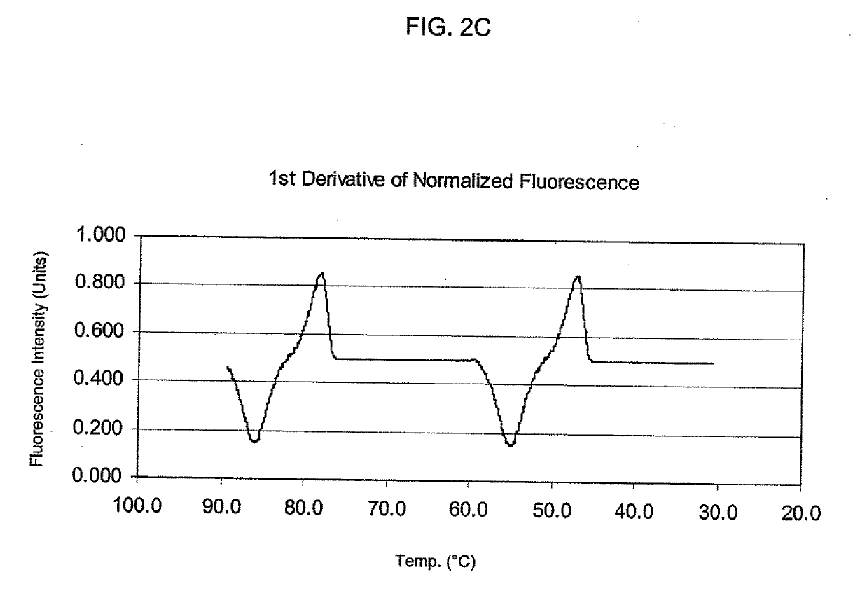

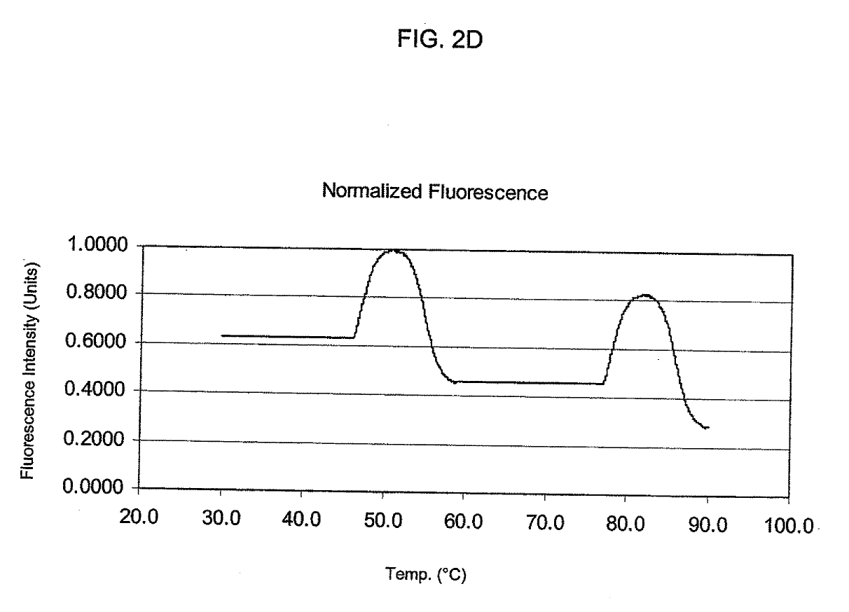

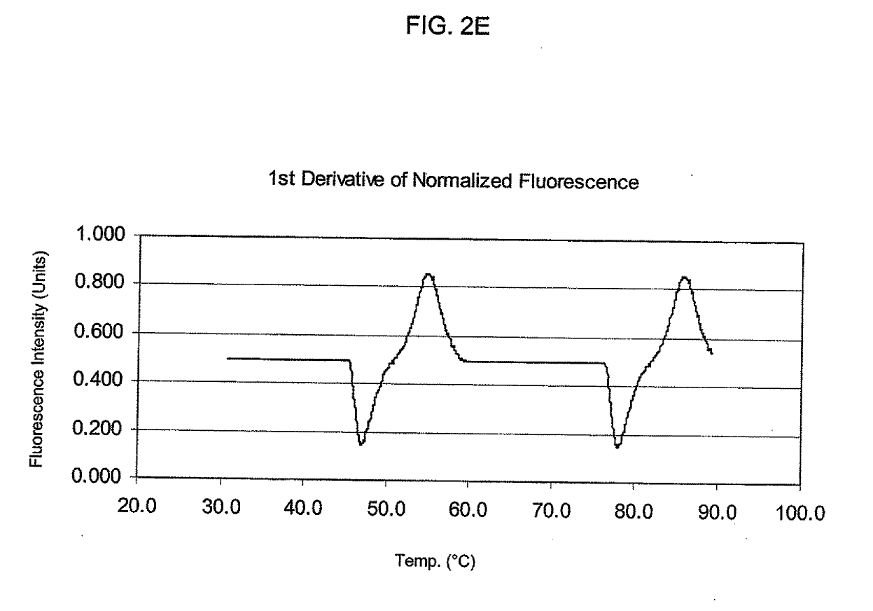

“Nucleobase” means those naturally occurring and those synthetic heterocyclic moieties commonly known to those who utilize nucleic acid or polynucleotide technology or utilize polyamide or peptide nucleic acid technology to thereby generate polymers that can hybridize to polynucleotides in a sequence-specific manner. Non-limiting examples of suitable nucleobases include: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Other non-limiting examples of suitable nucleobases include those nucleobases illustrated in FIGS. 2(A) and 2(B) of Buchardt et al. (WO 92/20702 or WO 92/20703).

“Nucleobase Polymer or Oligomer” refers to two or more nucleobases that are connected by linkages that permit the resultant nucleobase polymer or oligomer to hybridize to a polynucleotide having a complementary nucleobase sequence. Nucleobase polymers or oligomers include, but are not limited to, poly- and oligonucleotides (e.g., DNA and RNA polymers and oligomers), poly- and oligonucleotide analogs and poly- and oligonucleotide mimics, such as polyamide or peptide nucleic acids. Nucleobase polymers or oligomers can vary in size from a few nucleobases, from 2 to 40 nucleobases, to several hundred nucleobases, to several thousand nucleobases, or more.

“Polynucleotides or Oligonucleotides” refer to nucleobase polymers or oligomers in which the nucleobases are connected by sugar phosphate linkages (sugar-phosphate backbone). Exemplary poly- and oligonucleotides include polymers of 2′-deoxyribonucleotides (DNA) and polymers of ribonucleotides (RNA). A polynucleotide may be composed entirely of ribonucleotides, entirely of 2′-deoxyribonucleotides or combinations thereof.

“Polynucleotide or Oligonucleotide Analog” refers to nucleobase polymers or oligomers in which the nucleobases are connected by a sugar phosphate backbone comprising one or more sugar phosphate analogs. Typical sugar phosphate analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2′-deoxyribose or ribose, nucleobase polymers having positively charged sugar-guanidyl interlinkages such as those described in U.S. Pat. No. 6,013,785 and U.S. Pat. No. 5,696,253 (see also, Dagani 1995, Chem. & Eng. News 4-5:1153; Dempey et al., 1995, J. Am, Chem. Soc. 117:6140-6141). Such positively charged analogues in which the sugar is 2′-deoxyribose are referred to as “DNGs,” whereas those in which the sugar is ribose are referred to as “RNGs.” Specifically included within the definition of poly- and oligonucleotide analogs are locked nucleic acids (LNAs; see, e.g. Elayadi et al., 2002, Biochemistry 41:9973-9981; Koshkin et al., 1998, J. Am. Chem. Soc. 120:13252-3; Koshkin et al., 1998, Tetrahedron Letters, 39:4381-4384; Juniar et al., 1998, Bioorganic & Medicinal Chemistry Letters 8:2219-2222; Singh and Wengel, 1998, Chem. Commun., 12:1247-1248; WO 00/56746; WO 02/28875; and, WO 01/48190; all of which are incorporated herein by reference in their entireties).

“Polynucleotide or Oligonucleotide Mimic” refers to a nucleobase polymer or oligomer in which one or more of the backbone sugar-phosphate linkages is replaced with a sugar-phosphate analog. Such mimics are capable of hybridizing to complementary polynucleotides or oligonucleotides, or polynucleotide or oligonucleotide analogs or to other polynucleotide or oligonucleotide mimics, and may include backbones comprising one or more of the following linkages: positively charged polyamide backbone with alkylamine side chains as described in U.S. Pat. No. 5,786,461; U.S. Pat. No. 5,766,855; U.S. Pat. No. 5,719,262; U.S. Pat. No. 5,539,082 and WO 98/03542 (see also, Haaima et al., 1996, Angewandte Chemie Intl Ed. in English 35:1939-1942; Lesnick et al., 1997, Nucleosid. Nucleotid. 16:1775-1779; D′Costa et al., 1999, Org. Lett. 1:1513-1516 see also Nielsen, 1999, Curr. Opin. Biotechnol. 10:71-75); uncharged polyamide backbones as described in WO 92/20702 and U.S. Pat. No. 5,539,082; uncharged morpholino-phosphoramidate backbones as described in U.S. Pat. No. 5,698,685, U.S. Pat. No. 5,470,974, U.S. Pat. No. 5,378,841 and U.S. Pat. No. 5,185,144 (see also, Wages et al., 1997, BioTechniques 23:1116-1121); peptide-based nucleic acid mimic backbones (see, e.g., U.S. Pat. No. 5,698,685); carbamate backbones (see, e.g., Stirchak & Summerton, 1987, J. Org. Chem. 52:4202); amide backbones (see, e.g., Lebreton, 1994, Synlett. February, 1994:137); methylhydroxyl amine backbones (see, e.g., Vasseur et al., 1992, J. Am. Chem. Soc. 114:4006); 3′-thioformacetal backbones (see, e.g., Jones et al., 1993, J. Org. Chem. 58:2983) and sulfamate backbones (see, e.g., U.S. Pat. No. 5,470,967). All of the preceding references are herein incorporated by reference.

“Peptide Nucleic Acid” or “PNA” refers to poly- or oligonucleotide mimics in which the nucleobases are connected by amino linkages (uncharged polyamide backbone) such as described in any one or more of U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,451,968, 6,441,130, 6,414,112 and 6,403,763; all of which are incorporated herein by reference. The term “peptide nucleic acid” or “PNA” shall also apply to any oligomer or polymer comprising two or more subunits of those polynucleotide mimics described in the following publications: Lagriffoul et al., 1994, Bioorganic & Medicinal Chemistry Letters, 4: 1081-1082; Petersen et al., 1996, Bioorganic & Medicinal Chemistry Letters, 6: 793-796; Diderichsen et al., 1996, Tett. Lett. 37: 475-478; Fujii et al., 1997, Bioorg. Med. Chem. Lett. 7: 637-627; Jordan et al., 1997, Bioorg. Med. Chem. Lett. 7: 687-690; Krotz et al., 1995, Tett. Lett. 36: 6941-6944; Lagriffoul et al, 1994, Bioorg. Med. Chem. Lett. 4: 1081-1082; Diederichsen, U., 1997, Bioorganic & Medicinal Chemistry 25 Letters, 7: 1743-1746; Lowe et al., 1997, J. Chem. Soc. Perkin Trans. 1, 1: 539-546; Lowe et al., 1997, J. Chem. Soc. Perkin Trans. 11: 547-554; Lowe et al., 1997, I. Chem. Soc. Perkin Trans. 11:5 55-560; Howarth et al., 1997, I. Org. Chem. 62: 5441-5450; Altmann, K-H et al., 1997, Bioorganic & Medicinal Chemistry Letters, 7: 1119-1122; Diederichsen, U., 1998, Bioorganic & Med. Chem. Lett., 8:165-168; Diederichsen et al., 1998, Angew. Chem. mt. Ed., 37: 302-305; Cantin et al., 1997, Tett. Lett., 38: 4211-4214; Ciapetti et al., 1997, Tetrahedron, 53: 1167-1176; Lagriffoule et al., 1997, Chem. Eur. 1. '3: 912-919; Kumar et al., 2001, Organic Letters 3(9): 1269-1272; and the Peptide-Based Nucleic Acid Mimics (PENAMs) of Shah et al. as disclosed in WO 96/04000. All of which are incorporated herein by reference.

Some examples of PNAs are those in which the nucleobases are attached to an N-(2-aminoethyl)-glycine backbone, i.e., a peptide-like, amide-linked unit (see, e.g., U.S. Pat. No. 5,719,262; Buchardt et al., 1992, WO 92/20702; Nielsen et al., 1991, Science 254:1497-1500). A partial structure of N-(2-aminoethyl)-glycine PNA, a PNA suitable for use in the methods and compositions described herein is illustrated in structure (I), below:

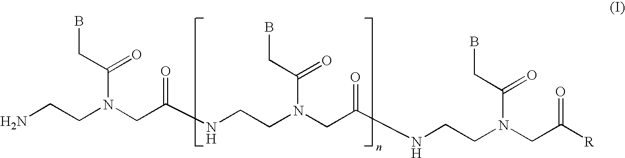

wherein:
(a) n is an integer that defines the length of the N-(2-aminoethyl)-glycine PNA;

each B is independently a nucleobase; and
R is —OR′ or —NR′R′, where each R′ is independently hydrogen or (C₁-C₆) alkyl, preferably hydrogen.

“Chimeric Oligo” refers to a nucleobase polymer or oligomer comprising a plurality of different polynucleotides, polynucleotide analogs and polynucleotide mimics. For example a chimeric oligo may comprise a sequence of DNA linked to a sequence of RNA. Other examples of chimeric oligos include a sequence of DNA linked to a sequence of PNA, and a sequence of RNA linked to a sequence of PNA.

“Signal Label” refers to a moiety that, when attached to a probe described herein, renders such a probe detectable using known detection methods, e.g., spectroscopic, photochemical, or electrochemiluminescent methods. Exemplary labels include but are not limited to fluorophores and chemiluminescent labels. Such labels allow direct detection of labeled compounds by a suitable detector, e.g., a fluorometer. In some embodiments, the label is a fluorogenic reporter dye detectable by a fluorometer and forms part of a reporter-quencher dye pair.

“Quencher Label” refers to a moiety capable of quenching the detectable signal produced by a signal label when positioned within quenching proximity thereto.

“Watson/Crick Base-Pairing” refers to a pattern of specific pairs of nucleobases and analogs that bind together through sequence-specific hydrogen-bonds, e.g. A pairs with T and U, and G pairs with C.

“Nucleoside” refers to a compound comprising a purine, deazapurine, or pyrimidine nucleobase, e.g., adenine, guanine, cytosine, uracil, thymine, 7-deazaadenine, 7-deazaguanosine, and the like, that is linked to a pentose at the 1′-position. When the nucleoside nucleobase is purine or 7-deazapurine, the pentose is attached to the nucleobase at the 9-position of the purine or deazapurine, and when the nucleobase is pyrimidine, the pentose is attached to the nucleobase at the 1-position of the pyrimidine, (see e.g., Kornberg and Baker, DNA Replication, 2nd Ed. (Freeman, San Francisco, 1992)). The term “nucleotide” as used herein, refers to a phosphate ester of a nucleoside, e.g., a triphosphate ester, wherein the most common site of esterification is the hydroxyl group attached to the C-5 position of the pentose. The term “nucleoside/tide” as used herein refers to a set of compounds including both nucleosides and nucleotides.

“Quench” refers to a measurable decrease in the quantity of a detectable signal produced by a signal label, regardless of the mechanism by which the measurable decrease occurs.

“Quenching Proximity” refers to the positions of a signal-quencher probe pair on a target sequence. To be in “quenching proximity” the signal probe and the quencher probe must hybridize in a configuration that positions the signal label sufficiently close to the quencher label such that a measurable decrease in the quantity of detectable signal produced by the signal label results.

“Annealing” or “Hybridization” refers to the base-pairing interactions of one nucleobase polymer with another that results in the formation of a double-stranded structure, a triplex structure or a quaternary structure. Annealing or hybridization can occur via Watson-Crick base-pairing interactions, but may be mediated by other hydrogen-bonding interactions, such as Hoogsteen base pairing.

### Various Exemplary Embodiments

Provided herein are compositions and methods for the multiplex analysis of polynucleotide samples. In some embodiments, methods for the multiplex analysis of polynucleotide samples by Tₘ (Tₘ multiplex analysis) using a plurality of signal-quencher nucleobase oligomer probe pairs bearing indistinguishable labels and having specified relative thermal melting temperatures are provided. For example, a detectable signal can be emitted (i.e., turned on) by a signal probe when it is hybridized to a region of a target sequence and is quenched (i.e., turned off) when a quencher probe hybridizes within quenching proximity to the signal probe. Each quencher probe can have a lower Tₘ than its corresponding signal probe, and the signal probe of each signal-quencher probe pair can have a lower Tₘ than the quencher probe of the preceding quencher-probe pair, except for the first signal probe, which can have the highest Tₘ of all signal and quencher probes used in the assay. By virtue of the specified relative Tₘs, as the temperature is increased or decreased through a temperature range including the Tₘs of the various probes, the signals produced by the signal probes turn on or turn off as their corresponding quencher probes either hybridize to or melt off the target sequence. Thus, the presence or absence of one or more target sequences in a polynucleotide sample may be assessed by the on or off state of signal as a function of temperature.

The multiplex assay may be used to analyze polynucleotide samples from many different sources. The sample may include a single polynucleotide suspected of having one or more different target sequences, or it may include a plurality of different polynucleotides, each of which may include none, one or a plurality of different target sequences.

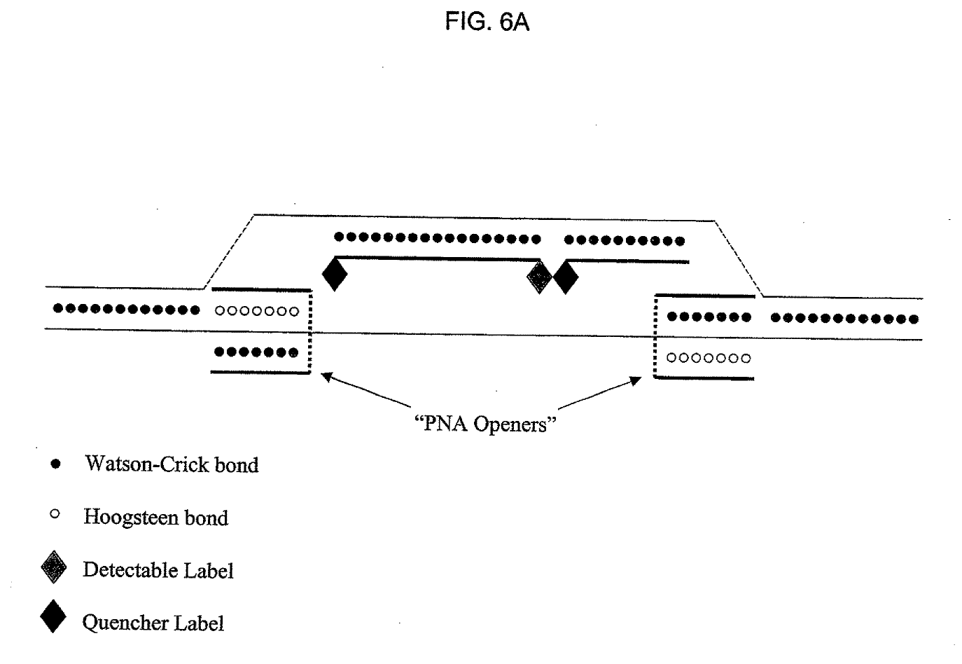

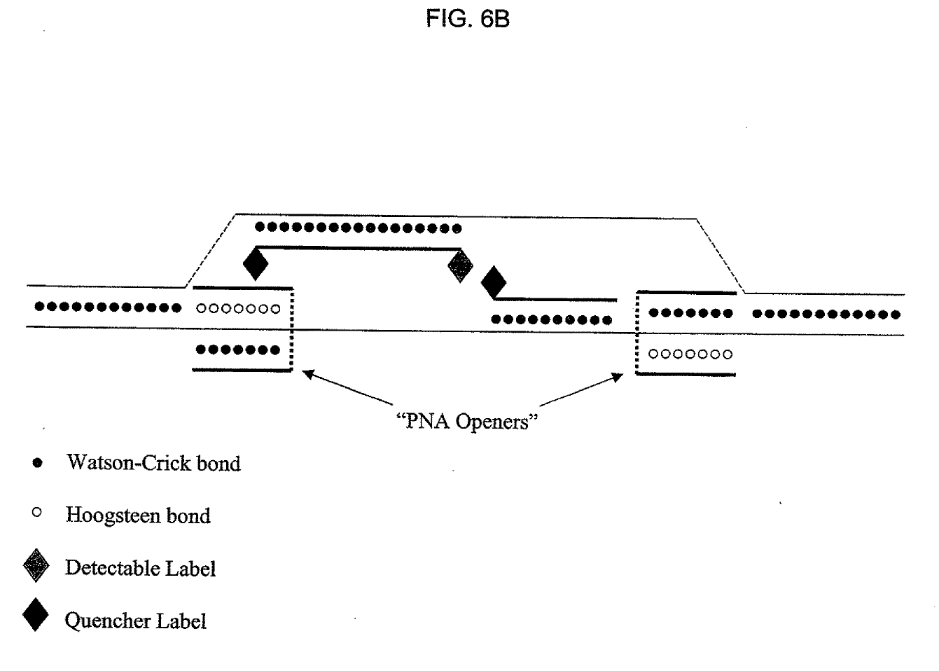

By “target sequence” herein is meant a nucleobase sequence on a polynucleotide sought to be detected. It is to be understood that the nature of the target sequence is not a limitation of the compositions and methods described herein. Each target sequence comprises a region of unique nucleobase sequence that may be used to discriminate one target sequence from another target sequence. In addition, each target sequence may also comprise a region of nucleobase sequence that is common to other target sequences and can not be used to discriminate one target sequence from another. The nucleobase sequences that comprise the target sequence may be on the same strand in a double-stranded polynucleotide (FIG. 6A) or on different strands in a double-stranded polynucleotide (FIG. 6B).

The polynucleotide comprising the target sequence may be provided from any source. For example, the target sequence may exist as part of a nucleobase polymer or oligomer, polynucleotide or oligonucleotide, polynucleotide or oligonucleotide analog, polynucleotide or oligonucleotide mimic, or chimeric oligo. The sample containing the target sequence may be provided from nature or it may be synthesized or supplied from a manufacturing process. The target sequence may be obtained from any source and amplified. For example, the target sequence can be produced from an amplification process, contained in a cell or organism or otherwise be extracted from a cell or organism. Examples of amplification processes that can be the source for the target sequence include, but are not limited to, Polymerase Chain Reaction (PCR), Ligase Chain Reaction (LCR), Strand Displacement Amplification (SDA; see, e.g., Walker et al., 1989, PNAS 89:392-396; Walker et al., 1992, Nucl. Acids Res. 20(7):1691-1696; Nadeau et al., 1999, Anal. Biochem. 276(2):177-187; and U.S. Pat. Nos. 5,270,184, 5,422,252, 5,455,166 and 5,470,723), Transcription-Mediated Amplification (TMA), Q-beta replicase amplification (Q-beta), Rolling Circle Amplification (RCA), Lizardi, 1998, Nat. Genetics 19(3):225-232 and U.S. Pat. No. 5,854,033), or Asynchronous PCR (see, e.g., WO 01/94638).

The signal and quencher probes of the present invention can be designed to form double-stranded hybrids with one strand of a polynucleotide or with a region of a polynucleotide that includes the target sequence. Polynucleotides that do not exist in a single-stranded state in the region of the target sequence(s) can be rendered single-stranded in such region(s) prior to detection or hybridization. For polynucleotides obtained via amplification, methods suitable for generating single-stranded amplification products are preferred. Non-limiting examples of amplification processes suitable for generating single-stranded amplification product polynucleotides include, but are not limited to, T7 RNA polymerase run-off transcription, RCA, Asymmetric PCR (Bachmann et al., 1990, Nucleic Acid Res., 18, 1309), and Asynchronous PCR (WO 01/94638). Commonly known methods for rendering regions of double-stranded polynucleotides single stranded, such as the use of PNA openers (U.S. Pat. No. 6,265,166), may also be used to generate single-stranded target sequences on a polynucleotide.

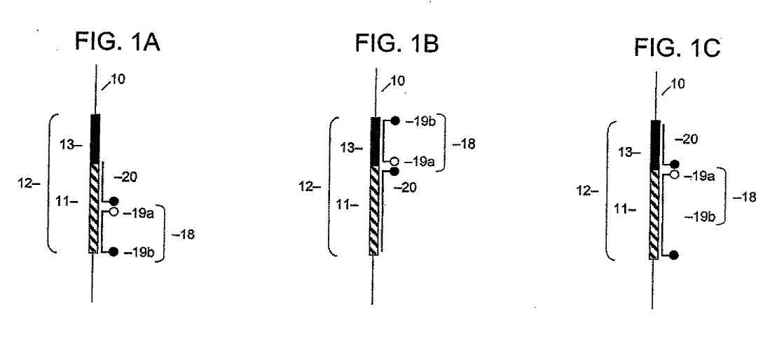

The nucleobase sequences of the signal and quencher probe pairs can be designed to be used together to detect a target sequence. FIG. 1A illustrates embodiments, in which the nucleobase sequences of the signal and quencher probes can be designed to hybridize to the region of the target sequence comprising a discriminating nucleobase sequence. By “discriminating nucleobase sequence” herein is meant a sequence that is unique to a given target sequence and can be used to discriminate that target sequence from another target sequence.

In other embodiments, the nucleobase sequence of the quencher probe can be designed to hybridize to the region of the target sequence comprising the discriminatory nucleobase sequence and the nucleobase sequence of the signal probe can be designed to hybridize to the region of the target sequence comprising the non-discriminatory nucleobase sequence. By “non-discriminatory” nucleobase sequence herein is meant a nucleobase sequence that is common to other target sequences. An exemplary embodiment is illustrated in FIG. 1B.

In yet other embodiments, the nucleobase sequence of the signal probe can be designed to hybridize to the region of the target sequence comprising the discriminatory nucleobase sequence and the nucleobase sequence of the quencher probe can be designed to hybridize to the region of the target sequence comprising the non-discriminatory nucleobase sequence. An exemplary embodiment is illustrated in FIG. 1C.

Although the above embodiments are depicted for a target sequence present on the same polynucleotide strand, a given signal-quencher probe pair may hybridize to different strands of a polynucleotide. FIG. 6B illustrates embodiments in which the target sequence is present on both strands of the polynucleotide. In these embodiments, the signal probe hybridizes to a portion of the target sequence located on one strand of the polynucleotide, while the quencher probe hybridizes to a portion of the target sequence located on the other strand of the polynucleotide.

The chemical composition of the signal and quencher probes is not critical to the success of the compositions and methods described herein. Virtually any nucleobase oligomer that is capable of hybridizing to a target polynucleotide in a sequence-specific manner may be used in the compositions and methods described herein. Thus, signal and quencher probes useful in the compositions and methods described herein include, but are not limited to, oligonucleotides, oligonucleotide analogs, oligonucleotide mimics such as PNAs and chimeric oligos, as defined above. In some embodiments, the signal and quencher probes can be resistant to degradation by nucleases (e.g., exonucleases and/or endonucleases). Nuclease-resistant probes include, by way of example and not limitation, oligonucleotide mimic probes such as PNA probes.

Although in many instances the signal and quencher probes of a specific signal-quencher probe pair will be of the same chemical composition (e.g., both DNA oligomers or both PNA oligomers), they need not be. Indeed, as will be discussed in more detail below, in some instances it may be desirable to utilize signal and quencher probes having different chemical compositions in order to achieve the necessary differential Tₘs.

Moreover, the chemical compositions of the various different signal and quencher probes may be the same or different. As a specific example, all of the signal probes may be DNA oligomers, all may be PNA oligomers, or some may be DNA oligomers and others PNA oligomers. Similarly, all of the quencher probes may be DNA oligomers, all may be PNA oligomers, or some may be DNA oligomers and some PNA oligomers.

Regardless of its chemical composition, the signal probe includes a reporter or signal label capable of producing a detectable signal when the signal probe is hybridized to a target sequence. The signal label may be a direct label, i.e., a label that itself is detectable or produces a detectable signal, or it may be an indirect label, i.e., a label that produces a detectable signal in the presence of another compound. Although the type of label is not critical to success, it is important that the label produce a detectable signal that can be quenched by a quencher probe hybridized in quenching proximity. Examples of suitable direct signal labels include, but are not limited to, fluorophores, chromophores, chemiluminescent moieties, etc. Examples of suitable indirect signal labels include, but are not limited to, enzymes capable of reacting with a substrate to produce a detectable signal (e.g., alkaline phosphatase, horseradish peroxidase, lysozyme, glucose-6-phosphate dehydrogenase, lactate dehydrogenase, urease, etc.), or other molecules or moieties that are capable of binding another label. For example, biotin can be detected using a streptavidin-chemiluminescent conjugate.

The quencher probe includes a quencher label capable of quenching the detectable signal produced by the signal label on the signal probe. Quenching occurs when the quencher probe is hybridized in close proximity to the signal probe, thereby bringing the quencher label sufficiently close to the signal label to result in a measurable decrease in the quantity of detectable signal produced by the signal label. For any given signal-quencher probe pair, quenching will be affected by factors such as the identity of the quencher and signal label, how the signal-quencher probe pair has been designed to hybridize to the target sequence, as well as the proximity of the signal probe to the quencher probe (i.e., whether or not the signal probe and quencher probe are contiguous or separated by one or more nucleotides). The identity of the quencher label can depend upon the identity of the signal label included on the signal probe, and will be apparent to those of skill in the art. For example, if the signal probe includes an indirect enzymatic label, the quencher label may be an inhibitor of the enzyme (see for example Saghatelian et al., 2003, J. Am. Chem. Soc., 125:344-345, describing a system comprising covalently associated inhibitor-DNA-enzyme modules for DNA detection; incorporated herein by reference in its entirety). If the signal label is a fluorophore, the quencher label may be a fluorophore, chromophore or other moiety capable of quenching the emission of the signal fluorophore (these types of signal-quencher label pairs are discussed in more detail, below).

The signal and quencher labels may be attached to the signal and quencher probes, respectively, at virtually any position, provided that the quencher label is able to quench the detectable signal produced by the signal label when the signal and quencher probes are hybridized to their respective regions or portions of the same target sequence. Thus, the signal and quencher labels may each be attached independently to a terminus, to a terminal or internal nucleobase or to the backbone of the signal and quencher probes.

In some embodiments, the signal and quencher labels are attached at or near a terminal residue of their corresponding signal and quencher probes (e.g., the 5′- or 3′-terminal nucleotide of an oligonucleotide probe or the amino- or carboxyl-terminal residue of a PNA probe). The label may be attached to the terminal residue at the nucleobase, or at the terminus (e.g., the 5′- or 3′-terminus of an oligonucleotide probe or the amino or carboxy terminus of a PNA probe). When attached to terminal residues, the signal and quencher labels can be positioned at opposite termini such that they are favorably oriented in space to allow for quenching when the signal and quencher probes are hybridized to the target sequence. For example, when the signal and quencher probes are oligonucleotides, if the signal label is attached to the 5′-terminal nucleotide, the quencher label should be positioned at the 3′-terminal nucleotide, and vice versa. For PNA signal and quencher probes, the positioning of the labels will depend upon whether the probes are designed to hybridize to the target sequence in an antiparallel or parallel orientation. For example, if both the signal and quencher probe are designed to hybridize to the target sequence with the same orientations, and the signal label is attached to the amino-terminal residue of the signal probe, then the quencher label should be attached to the carboxy-terminal residue of the quencher probe, and vice versa. If the signal and quencher probes are designed to hybridize to the target sequence with opposite orientations (i.e., one parallel and one antiparallel), the signal and quencher labels should be attached to the same type of terminal residue; that is, the signal label and quencher label should each be attached to the amino terminus of their respective probes or to the carboxy terminus of their respective probes.

The mechanism by which quenching occurs is not critical. Any mechanism by which quenching may occur may be used in the practice of the methods described herein. Quenching may occur via fluorescence resonance energy transfer (FRET), via non-FRET mechanisms such as collision or direct contact (see, e.g., Yaron et al., 1979, Analytical Biochemistry 95:228-235), by a combination of FRET and non-FRET mechanisms, or by a mechanism or mechanisms not yet understood.

Dye moieties capable of transferring energy from one moiety to another can be used in the methods described herein. In some embodiments, a plurality of dye moieties capable of transferring energy from one moiety to another can be used in the methods described herein. For example, three dye moieties may be used, where one member serves as the first donor, and the other dye moieties serve as acceptors/donors that can receive and transfer excitation energy. As will be appreciated by those of skill in the art, energy transfer cascades comprising multiple dyes can also be used in the methods described herein.

In some embodiments, dye pairs capable of transferring energy from the donor member of the pair to the acceptor member of the pair can be used as signal and quencher labels. Such dye pairs are well known in the art.

As one specific example, the quenching moiety may be a dye molecule capable of quenching the fluorescence of the signal fluorophores via the well-known phenomenon of FRET (also known as non-radiative energy transfer or Förster energy transfer). In FRET, an excited fluorophore (donor dye; in this instance the signal fluorophore) transfers its excitation energy to another chromophore (acceptor dye; in this instance the quencher). Such a FRET acceptor or quencher may itself be a fluorophore, emitting the transferred energy as fluorescence (fluorogenic FRET quencher or acceptor), or it may be non-fluorescent, emitting the transferred energy by other decay mechanisms (dark FRET quencher or acceptor). Efficient energy transfer depends directly upon the spectral overlap between the emission spectrum of the FRET donor and the absorption spectrum of the FRET quencher or acceptor, as well as the distance between the FRET donor and acceptor), as will be discussed in more detail, below.

Examples of signal and quencher labels that are FRET dye pairs are well known in the art, see for example, Marras et al., 2002, Nucleic Acids Res., 30(21) e122; Wittwer et al., 1997, Biotechniques 22:130-138; Lay and Wittwer, 1997, Clin. Chem. 43:2262-2267; Bernard et al., 1998, Anal. Biochem. 255:101-107; U.S. Pat. No. 6,427,156; and U.S. Pat. No. 6,140,054, the disclosures of which are incorporated herein by reference.

In some embodiments, the signal label of the signal probe is a fluorophore and the quencher label of the quencher probe is a moiety capable of quenching the fluorescence signal of the signal fluorophores. Fluorophores are known in the art. Examples of moieties capable of quenching fluorescence signals include Dabcyl, dabsyl BHQ-1, TMR, QSY-7, BHQ-2, blackhole quencher (Biosearch), and aromatic compounds with nitro or azo groups.

In another specific example, the quenching moiety may be a molecule or chromophore capable of quenching the fluorescence of the signal fluorophore via non-FRET mechanisms. For quenching via collision or direct contact, no spectral overlap between the signal fluorophores and quenching chromophore is required, but the signal fluorophore and quenching chromophore should be in close enough proximity of one another to collide.

As mentioned previously, the efficiency of energy transfer between donor (signal) and acceptor (quencher) labels can be dependent upon the distance between them. The distance between the donor and acceptor labels, depends on a number of factors, including the proximity with which the signal and quencher probes hybridize to one another. Thus, in some embodiments, the signal and quencher probes can be designed to hybridize contiguously to one another on a target sequence. The signal and quencher probes can be designed to hybridze non-contiguously to one another on a target sequence. For example, between 1 to 5 nucleobases can separate the signal probe from the quencher probe. Typically, the signal and quencher probes are designed to hybridize to the target sequence such that they are separated by zero or one nucleobase.

As will be recognized by skilled artisans, the lengths of the linkers used to attach the labels to the probes can be depend upon, among other factors, the point of attachment of the label to the probe (i.e., whether at a terminal nucleobase or terminal residue) and the proximity with which the signal and quencher probes hybridize to one another. For example, if the signal and quencher probes are designed to hybridize contiguously to one another on a target sequence and their corresponding labels are attached to juxtaposed terminal residues, relatively short linkers may be used. Signal and quencher probes designed to hybridize non-contiguously may require the use of longer linkers. All of these principles are well understood and skilled artisans will be able to routinely design labeled signal and quencher probes suitable for particular applications.

In some embodiments, the signal probe can be a self-indicating probe. As used herein, a “self-indicating” signal probe is a signal probe that produces little or no detectable signal when free in solution (i.e., unhybridized to a target sequence) and produces a detectable signal when hybridized to a target sequence. Alternatively, a self-indicating probe may produce a first detectable signal when free in solution and a second detectable signal distinguishable from the first detectable signal when hybridized to the target sequence. By virtue of these differential signals, a self-indicating probe is “off” when unhybridized and “on” when hybridized.

The nature of the differential signal of a self-indicating probe is not critical. All that is necessary is the ability to discriminate in some way the signal produced by the signal probe when hybridized and unhybridized to the target polynucleotide. For example, the differential signal may be an increase or decrease in signal intensity upon hybridization, a shift in emission spectrum upon hybridization, a change in fluorescence polarization, a change in electrochemical potential or a change in electrochemiluminescent state.

The use of fluorescent self-indicating signal probes has many advantages. One such advantage is low signal to noise ratio, as the background level of fluorescence signal is low because the probe produces little or no detectable signal when free in solution. Another advantage is that washing steps can be eliminated or minimized because the unhybridized probe produces little or no fluorescence signal when free in solution. Still another significant advantage of using self-indicating probes is that the assay can be carried out in a closed system thereby preventing contamination of the sample or future samples (see below).

Numerous self-indicating probes are known in the art that can be readily used or routinely adapted for use in the compositions and methods as self-indicating signal probes. In one specific example of a suitable self-indicating signal probe, the signal label is a moiety that produces a differential signal when in the presence of single-stranded versus double-stranded polynucleotides. Moieties having this property include, by way of example and not limitation, dyes that intercalate between base pairs of double-stranded polynucleotides such as double-stranded DNA, and dyes that bind the minor groove of double-stranded polynucleotides such as double-stranded DNA (MGB dyes). Numerous such intercalating and MGB dyes are known. Specific examples of suitable intercalating dyes include, but are not limited to, acridine orange, ethidium bromide, propidium iodide, hexium iodide, ethidium bromide homodimer, 3,3′-diethylthiadicarbocyanine iodide (Wilhelmsson et al., 2002, Nucleic Acids Res. 30(2) e3), SYBR® Green I and SYBR® Green II (Molecular Probes, Eugene, Oreg.) 7-aminoactinomycin D, actinomycin D (a non-fluorescent dye that changes absorbance upon intercalation) and other intercalating dyes available from Molecular Probes, Eugene, Oreg. (see, e.g., Molecular Probes Catalog, Sections 8.1, incorporated herein by reference).

Specific examples of suitable MOB dyes include, but are not limited to, bisbenziniide dyes such as Hoechst 332589, Hoechst 33342, and Hoechst 34580 and indole dyes such as DAPI (4′,6-diamino-2-phenylindole), as well as other MOB dyes available from Molecular Probes, Eugene, Oreg. (see, e.g., Molecular Probes Catalog, Section 8.1, supra).

These intercalating and MGB dyes may be linked to the signal probe using well-known techniques. Methods suitable for linking intercalating dyes to nucleobase oligomers such as signal probes are described, for example, in U.S. Pat. No. 4,835,263, the disclosure of which is incorporated herein by reference. Methods suitable for linking MOB dyes to nucleobase oligomers such as signal probes are described, for example, in U.S. Pat. Nos. 5,801,155, 6,492,346, and 6,486,308, the disclosures of which are incorporated herein by reference.

Another specific example of a self-indicating probe suitable for use as a self-indicating signal probe is a dual-label hairpin self-indicating probe. By “hairpin” is meant a construct comprising a single-stranded loop region and a double-stranded stem region. A dual-label hairpin probe is designed to have a donor molecule on one end and an acceptor molecule on the other. When unhybridized to a target sequence the acceptor molecule quenches the detectable signal produced by the donor molecule. When the hairpin probe hybridizes to a target sequence, the donor and acceptor become separated by a distance too great for efficient energy transfer, and the acceptor no longer efficiently quenches the signal produced by the donor. Thus, the hairpin probe is “off” when unhybridized to a target sequence (provided that the temperature of the solution is below the Tₘ of the hairpin stem region), and produces a detectable signal, i.e., is “on” when hybridized to the target sequence.

Hairpin self-indicating probes are well-known in the art (see, e.g., Tyagi et al., 1996, Nature Biotechnology 14:303-308; Nazarenko et al., 1997, Nucl. Acids Res. 25:2516-2521 for reviews see: Tan et al., 2000, Chem. Eur. J. 6:1107; Fang et al., 2000, Anal. Chem. 72:747 A; all of which are incorporated herein by reference) and have a nucleobase sequence capable of adopting a hairpin conformation in solution. In some embodiments, the hairpin probes include a FRET donor on one end (e.g., 3′-terminus) and a FRET acceptor at the other end (e.g., 5′-terminus) such that when the probe is in the hairpin conformation, the FRET acceptor quenches the detectable signal produced by the FRET donor. In other embodiments, non-FRET donor and acceptors are used (see U.S. Pat. No. 6,150,097; incorporated herein by reference). A hairpin self-indicating probe can be made entirely from PNA (see U.S. Pat. No. 6,355,412; incorporated herein by reference in its entirety).

Another specific example of a self-indicating probe suitable for use as a self-indicating signal probe is a linear dual-label probe. As used herein, “linear” refers to a probe that assumes a conformation that is not a hairpin conformation. However, the term “linear” is not intended to imply that the probe does not contain secondary or tertiary structure. Thus, a linear dual-label probe may be linear or assume a conformation that is not a hairpin conformation. Like hairpin probes, dual-label linear probes include a donor and an acceptor. Also like hairpin probes, dual-label linear probes can remain substantially quenched until hybridized to a target sequence. A variety of different types of dual-label linear probes suitable for use as self-indicating probes are known in the art, and include, by way of example and not limitation, the dual-label DNA probes commonly referred to in the art as TaqMan® probes (see U.S. Pat. Nos. 5,210,015, 6,258,569, and 6,503,720); the dual-label PNA probes described in Kuhn et al., 2002, J. Am. Chem. Soc. 124(6):1097-1103 (as well as the references cited therein), and are also described in U.S. Pat. No. 6,485,901 (as well as the references cited therein), the disclosures of which are incorporated herein by reference in their entirety.

In embodiments employing hairpin and linear dual-label self-indicating signal probes, the signal label of the signal probe corresponds to the donor of the dual labeled probe. The acceptor may be the same as the quencher label of the signal probe's corresponding quencher probe, or it may be different. An example of a dye that is recognized in the art as a universal acceptor dye because it can quench fluorescence signals from a number of different donor dyes without regard to spectral overlap is dabcyl. Selection of suitable signal labels and acceptors, as well as positions and linkers suitable for their attachment to the signal probe will be apparent to those of skill in the art.

Suitably labeled signal and quencher probes may be synthesized using routine methods. For example, methods of synthesizing oligonucleotide probes are described in U.S. Pat. No. 4,973,679; Beaucage, 1992, Tetrahedron 48:2223-2311; U.S. Pat. No. 4,415,732; U.S. Pat. No. 4,458,066; U.S. Pat. No. 5,047,524 and U.S. Pat. No. 5,262,530; all of which are incorporated herein by reference in their entirety. The synthesis may be accomplished using automated synthesizers available commercially, for example the Model 392, 394, 3948 and/or 3900 DNA/RNA synthesizers available from Applied Biosystems, Foster City, Calif.

Methods of synthesizing labeled oligonucleotide probes are also well-known. As a specific example see WO 01/94638 (especially the disclosure at pages 16-21), the disclosure of which is incorporated herein by reference in its entirety.

Methods of synthesizing labeled oligonucleotide analog probes are also well-known. See for example U.S. Pat. No. 6,479,650 and U.S. Pat. No. 6,432,642, both of which are incorporated herein by reference in their entirety.

Methods for the chemical assembly of PNAs are also well known (See: U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053 and 6,107,470; all of which are incorporated herein by reference in their entireties). As a general reference for PNA synthesis methodology also see: Nielsen et al., Peptide Nucleic Acids; Protocols and Applications, Horizon Scientific Press, Norfolk England (1999).

Non-limiting methods for labeling PNAs are described in U.S. Pat. No. 6,110,676, U.S. Pat. No. 6,280,964, WO 99/22018, now issued as U.S. Pat. No. 6,355,421, WO 99/21881, now issued as U.S. Pat. No. 6,485,901, WO 99/37670, now issued as U.S. Pat. No. 6,326,479, and WO 99/49293, now issued as U.S. Pat. No. 6,361,942, the examples section of this specification or are otherwise well known in the art of PNA synthesis and peptide synthesis. Methods for labeling PNA are also discussed in Nielsen et al., Peptide Nucleic Acids; Protocols and Applications, Horizon Scientific Press, Norfolk, England (1999). Non-limiting methods for labeling the PNA oligomers that can be used as signal and quencher probes are as follows. Because the synthetic chemistry of assembly is essentially the same, any method commonly used to label a peptide can often be adapted to effect the labeling of a PNA oligomer.

The synthesis, labeling and modification of PNA chimeras can utilize methods known to those of skill in the art as well as those described above. A suitable reference for the synthesis, labeling and modification of PNA chimeras can be found in WIPO published patent application number WO 96/40709, now issued as U.S. Pat. No. 6,063,569, incorporated herein by reference in its entirety. Moreover, the methods described above for PNA synthesis and labeling often can be used for modifying the PNA portion of a PNA chimera. Additionally, well known methods for the synthesis and labeling of nucleic acids can often be used for modifying the oligonucleotide portion of a PNA chimera. Exemplary methods can be found in U.S. Pat. No. 5,476,925, U.S. Pat. No. 5,453,496, U.S. Pat. No. 5,446,137, U.S. Pat. No. 5,419,966, U.S. Pat. No. 5,391,723, U.S. Pat. No. 5,391,667, U.S. Pat. No. 5,380,833, U.S. Pat. No. 5,348,868, U.S. Pat. No. 5,281,701, U.S. Pat. No. 5,278,302, U.S. Pat. No. 5,262,530, U.S. Pat. No. 5,243,038, U.S. Pat. No. 5,218,103, U.S. Pat. No. 5,204,456, U.S. Pat. No. 5,204,455, U.S. Pat. No. 5,198, U.S. Pat. No. 540, U.S. Pat. No. 5,175,209, U.S. Pat. No. 5,164,491, U.S. Pat. No. 5,112,962, U.S. Pat. No. 5,071,974, U.S. Pat. No. 5,047,524, U.S. Pat. No. 4,980,460, U.S. Pat. No. 4,923,901, U.S. Pat. No. 4,786,724, U.S. Pat. No. 4,725,677, U.S. Pat. No. 4,659,774, U.S. Pat. No. 4,500,707, U.S. Pat. No. 4,458,066 and U.S. Pat. No. 4,415,732, all of which are incorporated herein by reference in their entireties.

The basic principles of Tₘ multiplexing are illustrated in FIG. 2A, with reference to an embodiment employing a polynucleotide 10 having two different target sequences 12, 14 and three sets of signal-quencher probe pairs 16, 22, 28. Each target sequence includes a region of discriminating sequence 11, 15 and a region of non-discriminating sequence 13, 17. The first signal-quencher probe pair comprises first signal probe 18 and first quencher probe 20, the second comprises second signal probe 24 and second quencher probe 26 and the third pair comprises third signal probe 30 and optional third quencher probe 32. As illustrated, each signal probe 18, 24, 30 is a self-indicating signal probe and comprises a signal label (represented by 19a and quencher dye (e.g., FRET acceptor), represented by 19b. Alt of the quencher probes 20, 26, 32 include the same quencher label, which is the same as the quencher dye of self-indicating signal probes 18, 24, 30. In the embodiment illustrated, the signal probes 18, 24, 30 are designed to hybridize to the discriminating region of a target sequence, and the quencher probes 20, 26, 32 are designed to hybridize to the non-discriminating region of a target sequence.

As illustrated in FIG. 2A, for Tₘ multiplexing, the various signal and quencher probes are designed to have specified relative Tₘs: Tₘ (first signal probe 18)>Tₘ (first quencher probe 20)>Tₘ (second signal probe 24)>Tₘ (second quencher probe 26)>Tₘ (third signal probe 30)>Tₘ (third quencher probe 22), and so forth. Although the range between the lowest and highest probe Tₘ can vary, in some embodiments the range is between about 20-95° C., with a range of from about 30-85° C. being more preferred.

The Tₘ difference between any two successive probes (e.g., first signal probe 18 and first quencher probe 20, first quencher probe 20 and second signal probe 24, etc., referred to herein as ΔTₘ^{probe}) may also vary. In some embodiments, the ΔTₘ^{probe} is at least 5° C., typically ranging from about 5-10° C. The ΔTₘ^{probe} intervals may all be the same, or they may vary.

The Tₘ difference between any two successive signal-quencher probe pairs may also vary. As used herein, the Tₘ of a specific signal-quencher probe pair is the Tₘ of the signal probe. Thus, the difference between two successive probe-pairs may be designated as ΔTₘ^{signal probe}. In some embodiments, the ΔTₘ^{signal probe} is at least about 7-15° C., typically ranging from about 10-15° C. The ΔTₘ^{signal probe} intervals may all be the same, or they may be different.

As will be recognized by skilled artisans, the number of signal-quencher probe pairs that may be included in a single multiplex Tₘ assay is in part dependent upon the ΔTₘ^{probe} and ΔTₘ^{signal probe} intervals selected. Additional degrees of complexity in a single multiplex assay may be achieved by using distinguishable signal probe labels, such as signal labels that fluoresce at different colors, as will be described in more detail, below.

As is well-known in the art, the Tₘ of a specified probe is dependent upon external factors (e.g., salt concentration, pH, etc.) and internal factors (e.g., probe concentration, probe length, GC content, nearest neighbor interactions, etc.) (see, e.g., Wetmur, 1991, Crit. Rev. Biochem. Mol. Biol. 26:227-259; Wetmur, 1995, In: Molecular Biology and Biotechnology, Meyers, Ed., VCH, New York, pp. 605-608; Brown et al., 1990, J. Mol. Biol. 212:437-440; Gaffney et al., 1989, Biochemistry 28:5881-5889). Mismatches between a probe and a target sequence can cause a decrease in the probe Tₘ (see, e.g., Guo et al., 1997, Nat. Biotechnol. 15:331-335; Wallace et al., 1979, Nucleic Acids Res. 6:3543-3557). The type of mismatch can also impact the amount of decrease in probe Tₘ. Mismatches that are relatively stable, e.g., G-T mismatches, are known to decrease a DNA probe Tₘ by 2-3° C. (see, e.g., Bernardet et al., 1998, Anal. Biochem. 255:101-107), whereas less stable C-A mismatches are known to shift a DNA probe Tₘ by 8-10° C. (see, e.g., Lay et al., 1997, Clin. Chem. 43:2262-2267; Bernard et al., 1998, Am. J. Pathol. 153:1055-1061). The position and number of mismatches are also known to affect probe Tₘ (see, e.g., Wallace et al., 1979, supra). Accordingly, the percent of sequence identity of a probe with a target sequence can directly impact the temperature at which the probe will dissociate or melt from the complementary strand of the target sequence. The greater the sequence difference or mismatch between the probe and the target sequence the lower Tₘ of the probe. Thus, for example, a probe having a sequence that is perfectly complementary to a target sequence will dissociate at a higher temperature than a probe having a sequence that includes one or more mismatches.

The Tₘ of a specified probe may also be dependent upon its backbone composition. For example, oligonucleotide probes such as DNA and RNA oligos have negatively charged phosphodiester backbones. When hybridized to a target sequence such as a cDNA strand, which also has a negatively charged phosphodiester backbone, the negative charges repel one another. In contrast, several oligonucleotide mimics, such as PNA nucleobase oligomers, can have neutral backbones that are not electrostatically repelled when hybridized to DNA and/or RNA polynucleotides. Some nucleobase oligomers, such as nucleobase oligomers including sugar-guanidyl interlinkages, are positively charged and experience electrostatic attraction when hybridized to a target DNA or RNA polynucleotide.

All of the above factors, whether external or internal, may be used to design signal and quencher probes having appropriate Tₘs for particular multiplex applications. In some embodiments, the nucleobase sequences of the signal and quencher probes are designed to be completely complementary to a region of a target sequence. In these embodiments, the Tₘs of the probes may be adjusted or modified as necessary by adjusting the other factors discussed above.

The Tₘ of a probe may also be adjusted or modified by incorporating one or more conformationally locked nucleotides (LNA nucleotides) into the probe sequence. Depending on the identity of the LNA nucleotide, the Tₘ of the probe can be increased between 2 to 5 degrees per LNA nucleotide. For example, the Tₘ value of a probe containing the conformationally locked nucleotide bicyclic thymidine (T) can be increased in the range of 2.0-3.5 degrees per modification. Similarly, the Tₘ value of a probe sequence containing bicyclic cytidine (C) can be increased by 2 degrees or more per modification. Greater increases can be achieved by replacing more than one nucleotide with an LNA nucleotide. See, e.g., U.S. Pat. No. 6,503,566, WO 99/14226, and WO 00/56916; all of which are incorporated herein by reference in their entireties.

The Tₘ of a probe may be further adjusted or modified by the attachment of one or more duplex binding moieties (DBM) to the probe. As used herein, “duplex binding moiety” or “DBM” refers to a molecule that binds double-stranded polynucleotides. When included in a signal or quencher probe, the DBM binds the duplex formed by the hybridized probe, thereby stabilizing the hybrid resulting in an increased Tₘ. DBMs suitable for use include, but are not limited to, intercalating dyes (discussed previously) and minor groove binding (MGB) moieties. The MGB moieties include MGB dyes (discussed previously), as well as non-fluorescent molecules that bind the minor groove of double-stranded polynucleotides. Non-fluorescent MGB moieties suitable for use include, but are not limited to, netropsin, distamycin and lexitropsin, mithramycin, chromomycin A3, olivomycin, anthramycin, sibiromycin, as well as further related antibiotics and synthetic derivatives, diarylamidines such as pentamidine, stilbamidine and berenil, CC-1065 and related pyrroloindole and indole polypeptides, and a number of oligopeptides consisting of naturally occurring or synthetic amino acids.

Additional suitable MGB moieties, as well as chemistries, linkers and suitable positions for their attachment are described in U.S. Pat. Nos. 6,492,346 and 6,486,308, the disclosures of which are incorporated herein by reference in their entireties.

If a fluorescent DBM is included in a signal probe which is not the signal label, care should be taken to insure that the emissions spectrum of the DBM is distinguishable (spectrally resolvable) from that of the signal label. Care should also be taken to insure that such DBMs included in signal probes do not quench the signal produced by the signal label when the signal probe is hybridized to a target sequence.

Referring again to FIG. 2A, target polynucleotide 10 is contacted with signal-quencher probe pairs 16, 22, 28. The conditions under which the target sequences (12 and 14) and probe pairs are contacted (e.g., target/probe concentrations, salt concentration, pH, etc.), may vary, and may depend upon the conditions at which the Tₘs of the signal and quencher probes were calculated and/or measured empirically. Ideally, the conditions under which the target sequences and probe pairs are contacted will be the same as those used to calculate and/or measure empirically the Tₘs of the signal and quencher probes. Those skilled in the art are well versed in selecting hybridization conditions suitable for particular applications.

Hybridization variables that may be varied to optimize hybridization conditions for the probes and target sequences of the present invention include, target/probe concentrations, signal/quencher probe concentrations, salt concentration, pH, as well as other components of the hybridization buffer. Destabilizing agents such as formamide, may be added to the buffer. Those skilled in the art are well versed in selecting the appropriate hybridization variables to vary to optimize hybridization conditions for particular applications.

Once the polynucleotide sample 10 has been contacted with the signal-quencher probe pairs, the detectable signal produced by the signal labels of the signal probes is monitored as a function of temperature. The detection system used will depend upon the nature of the detectable signal produced by the signal probe label, and will be apparent to those of skill in the art. Devices for measuring emissions from fluorescent signal labels (at one or more wavelengths) are available commercially, as are devices for measuring emissions from fluorescent signal labels (at one or more wavelengths) at varying temperatures. Such devices include, by way of example and not limitation, the LightCycler™ instrument available from Roche (formerly from Idaho Technologies) and Prism™ 7700, 7900, 7000 Sequence detector instruments available from Applied Biosystems (Foster City, Calif.).

In some embodiments, the emission at the signal label of the signal probe is monitored, preferably at or around its maximum emission wavelength, as a function of decreasing temperature. The measurements may be made in a step-wise fashion by obtaining the emission at a first temperature, repeating the measurement at a lower temperature, and so forth. Alternatively, the emission measurements may be monitored continuously or at discrete temperature points as the temperature is decreased downward. When the emission measurements are monitored continuously, the temperature decreases at an approximate rate of 100-10,000 msec. When the emission measurements are monitored at discrete temperature points, the temperature is measured at a rate in the range of about 0.01-5° C./minute, or more preferably in the range of about 0.01-1° C./minute, 0.1-1° C./minute, or 0.5-1° C./minute.

Whether monitored continuously or at discrete temperatures, the detectable signal produced by the signal probes is monitored over a temperature range that includes the Tₘ of the probe with the highest Tₘ (typically the first signal probe) and the Tₘ of the probe with the lowest Tₘ (typically the nth signal probe or optional nth quencher probe). Preferably, the detectable signal is monitored from an initial temperature that is high enough to insure that all of the signal and quencher probes are unhybridized to a final temperature that is low enough to insure that all of the signal and quencher probes are hybridized. For signal-quencher probe pairs having Tₘs that range from 30-85° C., the detectable signals of the signal probes may be monitored over a temperature range of 95-20° C.

Referring again to FIG. 2A, at the initial temperature, which is above the Tₘ of first signal probe 18 (Panel A), all of the probes are unhybridized and no signal is detected, because all of the signal probes, 18, 24 and 30, are self indicating (i.e., all signal probes are “off”). At a temperature between the Tₘ of first signal probe 18, and first quencher probe 20, signal probe 18 hybridizes to a complementary discriminating region 11 of target sequence 12 on polynucleotide 10 (Panel B). At this temperature, the signal label 19a produces a detectable signal (turns “on”; indicated by 19c). At a temperature between the Tₘ of first quencher probe 20 and second signal probe 24, first quencher probe 20 hybridizes to a complementary non-discriminating region 13 of target sequence 12 on polynucleotide 10, thereby quenching (turning “off”) the signal produced by signal label 19a of first signal probe 20 (Panel C). At a temperature between the Tₘ of second signal probe 24 and second quencher probe 26, hybridization of second signal probe 24 to polynucleotide 10 would occur if polynucleotide 10 included a region of target sequence complementary to second signal probe 20; however, in the illustrated example, it does not (Panel D). Therefore, second signal probe 20 remains unhybridized and does not produce a detectable signal (remains “off”). In the illustrated example, at a temperature between the Tₘ of second quencher probe 26 and third signal probe 30 (Panel E), second quencher probe 26 also remains unhybridized. At a temperature between the Tₘ of third signal probe 30 and optional third quencher probe 32 (Panel F), third signal probe 30 hybridizes to complementary discriminating region 17 of target sequence 14 of polynucleotide 10 and gets turned “on.” If third signal-probe pair 28 includes an optional third quencher probe 32, the temperature may be lowered below the Tₘ of third quencher probe 32 (Panel G), which will hybridize to its complementary non-discriminating region 15 of target sequence 14 on polynucleotide 10, turning “off” the detectable signal of third signal probe 30.

The turning “off” and turning “on” of the detectable signal of each signal probe can be depicted by plotting detectable signal intensity versus temperature (see, e.g., FIG. 2B), the result of which is referred to herein as a multiplex Tₘ curve (or signal profile). Each signal detected at a specified temperature is indicative of the presence of a specific target sequence. Further, the first derivative of the signal profile can be calculated and illustrated in a graph such that the turning “on” of a detectable signal is depicted as a decrease or valley and the turning “off” of the signal is depicted as an increase or peak, at a specific temperature (see, e.g., FIG. 2C). One skilled in the art would know how to plot a signal profile and calculate its first derivative. Further, when fluorescent signal labels are used, most spectrofluorometers have software and a computer having such capabilities, and can be programmed to perform such an analysis automatically at predetermined intervals.

Alternatively, the detectable signals of the signal probes may be monitored as a function of increasing temperature starting at a temperature below the lowest probe Tₘ and ending with a temperature above the highest probe Tₘ. Again, the measurements may be made in a step-wise fashion by obtaining the emission at a first temperature, repeating the measurement at a higher temperature, and so forth. Alternatively, the emission measurements may be monitored continuously at an approximate rate of 100-10,000 msec or at discrete temperature points as the temperature is increased upwards, for example at a rate in the range of about 0.01-5° C./minute, or more preferably in the range of about 0.01-1° C./minute, 0.1-1° C./minute, or 0.5-1° C./minute. The sequence of hybridization is the reverse of that depicted in FIG. 2A; that is beginning with FIG. 2A, Panel G and ending with FIG. 2A Panel A. Thus, at the start of the analysis all complementary probes are hybridized to the targets, and all signal probes are turned “off.” As the temperature is increased, third quencher probe 32 melts off, permitting third signal probe 30 to turn “on,” and so forth until all of the probes are melted off (Panel A), and no signal is detected. The signal profile and first derivative of the signal profile obtained from proceeding from Panel G to Panel A are depicted in FIGS. 2D and 2E, respectively. It should be noted that if optional third quencher probe 32 is not used, the sequence of events begins with FIG. 2A, Panel F and proceeds through FIG. 2A, Panel A.

Accordingly, by increasing (or ramping up) the temperature or by decreasing (or ramping down) the temperature during the assay, a signal corresponding to the presence of target sequences can be detected as a function of temperature. Multiple probes can therefore be used to detect one or more target sequences on a single polynucleotide or on multiple polynucleotides. Because the signal corresponding to the presence of a target sequence is detected as a function of temperature, in some embodiments, it is not necessary to use distinguishable or spectrally resolvable signal labels for detection of multiple and different target sequences. The same signal label can be used on multiple signal probes because the probes each have a different Tₘ and the signal corresponding to the presence of a target sequence is detected as a function of temperature.

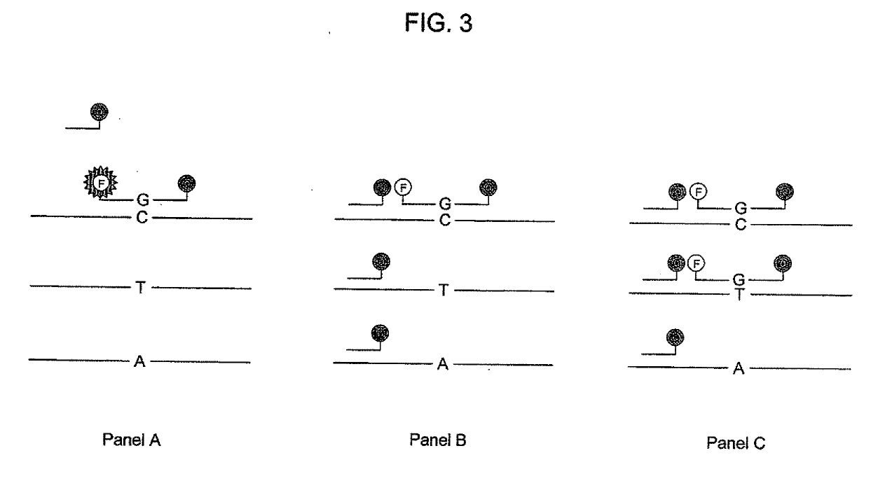

Tₘ multiplexing utilizing signal-quencher probe pairs effectively increases the specificity of the signal probe, as mismatched hybridizations are not reported. This significant advantageous feature is illustrated in FIG. 3. In FIG. 3, a signal-quencher probe pair is contacted with a polynucleotide sample that includes single nucleotide polymorphisms. At a temperature between the Tₘs of the signal and quencher probes, the signal probe hybridizes to its complementary sequence and produces a detectable signal (Panel A). At a temperature below the Tₘ of the signal probe, but above the Tₘ of a mismatched hybridization (Panel B), the quencher probe hybridizes to all three polymorphic targets, and quenches the signal of the hybridized signal probe. At temperatures below the Tₘ of a mismatched hybridization (Panel C), the signal probe hybridizes to one of the polymorphic targets (or more, depending upon the Tₘs of the mismatches). However, since the quencher probe is also hybridized to the polymorphic targets at this temperature, the mismatched signal probe does not produce a detectable signal—its signal is quenched by the adjacently hybridized quencher probe. By virtue of the use of quencher probes, mismatched hybridization events are not observed. As a consequence, the specificity of the signal probe is effectively increased. Accurate sequence analysis may be obtained without interference from the presence of polymorphic targets. The use of additional signal probes complementary to the different polymorphic targets bearing different, distinguishable labels would permit the accurate identification of all three polymorphic target sequences in a single assay.

As a person of skill in the art will appreciate, if the signal probe is not a self-indicating probe, detection of the signal will require multiple wash steps or the use of a continuous flow system to remove any detectable signal probe that is not hybridized to a target sequence of interest.

As evidenced by the above discussion, the complexity of the number of sequences that may be simultaneously identified or analyzed in a single multiplex assay may be increased by the use of both Tₘ multiplexing and signal multiplexing. As used herein, “signal multiplexing” refers to multiplexing accomplished on the basis of the detectable signals produced by the signal probes. For signal multiplexing, at least some of the signal probes include signal labels that produce detectable signals that are distinguishable from the others. In this vein, the presence or absence of a target sequence correlates with the presence or absence of a particular detectable signal. Such signal multiplexing is commonly employed in conventional SNP polynucleotide assays by labeling probes complementary to the different polymorphs with fluorophores that emit different, spectrally resolvable emissions signals (i.e., the probes are labeled with fluorophores of different colors).

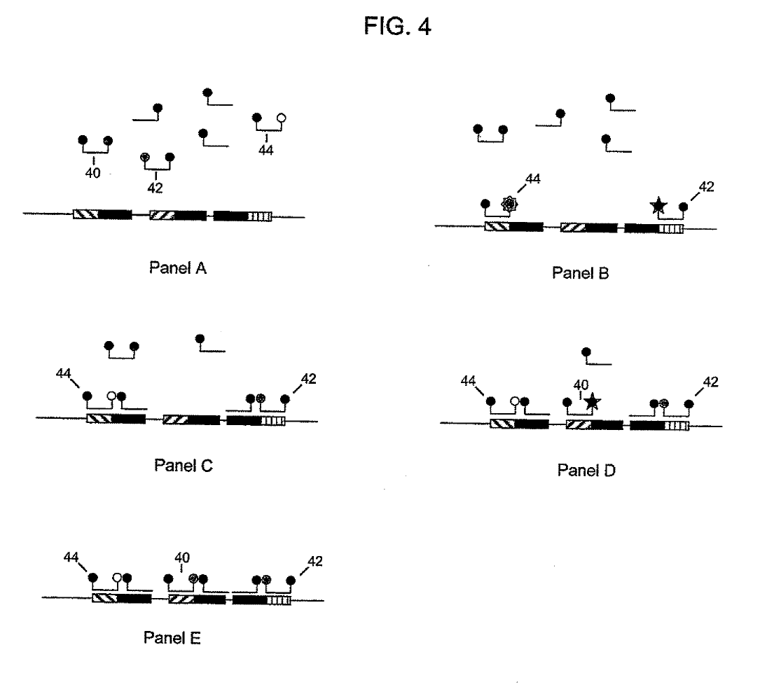

An example of dual Tₘ and signal multiplexing is illustrated in FIG. 4 with reference to an embodiment that employs two Tₘ multiplex signal-quencher probe pairs in which each signal probe 40, 42 is labeled with a fluorophore that emits a first color, and one Tₘ multiplex signal-quencher probe pair in which the signal probe 44 is labeled with a fluorophore that emits a second, spectrally resolvable color. In the illustrated embodiment, all of the signal probes are self-indicating signal probes. Moreover, all of the quencher probes are labeled with the same quencher label, although skilled artisans will recognize that the choice of quencher labels will depend upon the specific signal labels selected. In Panel A, the temperature is above the Tₘs of all of the probes. As a consequence, none of the signal probes produces a detectable signal. In Panel B, the temperature is below the Tₘ of signal probes 42 and 44, but above the Tₘs of all of the quencher probes. At this temperature, signal probe 42 emits a signal of a first color, and signal probe 44 emits a signal of a second color. Although both signals are present at the same temperature, they may be resolved on the basis of their different colors. In Panel C, as the temperature is lowered, the signals of probes 42 and 44 are quenched by the hybridization of their corresponding quencher probes. At an even lower temperature (Panel D), signal probe 40 hybridizes to its complementary target sequence and emits a signal of a first color. Although signal probes 40 and 42 emit signals of the same color, they are distinguishable from one another on the basis of their differential Tₘs. Finally, at an even lower temperature (Panel E), the quencher probe corresponding to signal probe 40 hybridizes to its complementary target sequence and quenches the signal of signal probe 40. As evident from FIG. 4, numerous target sequences may be analyzed by obtaining signal profiles corresponding to each of the two different, distinguishable signals.

This dual Tₘ and color multiplexing is limited only by the number of distinguishable labels available. Although FIG. 4 illustrates the use of only a single second-color signal-quencher probe pair, skilled artisans will recognize that any number of Tₘ multiplex signal-quencher probe pairs may be used for each distinguishable label, limited only by the number of signal-quencher probe pairs that can be distinguished over a specified temperature range. Moreover, as illustrated in FIG. 4, signal probes labeled with different distinguishable labels (e.g. colors) may have the same Tₘs, or they may have different Tₘs. Likewise, their corresponding quencher probes may have Tₘs that are the same as or different from those corresponding to the differently labeled signal probes.

The Tₘ and dual signal-Tₘ multiplex assays of the invention find use in virtually any type of hybridization-based assay useful for analyzing or detecting polynucleotide sequences. In certain embodiments, the Tₘ and dual signal-Tₘ multiplex assays can be used as an end point analysis of an amplification reaction. In such assays, the signal-quencher probe pairs may be included in the amplification reaction during the amplification, or may be added to the reaction mixture at the completion of amplification. When included in the amplification reaction, the signal and quencher probes are preferably nucleobase polymers that cannot be acted on by enzymes used in the amplification reaction (e.g., PNA oligomers). Following amplification, the detectable signals of the signal labels are monitored as a function of temperature, as previously described. Instruments suitable for carrying out amplifications followed by Tₘ and/or dual Tₘ-signal multiplex analysis preferably include an instrumentation platform having a thermal cycler, computer, a light source for excitation of reporter dyes (e.g., a laser or other broad spectrum light source with tunable filters), optics for collection of fluorescence excitation and emission data, and software for data acquisition and analysis. An example of an amplification and detection system suitable for use in the methods of the present invention includes, but is not limited to, the ABI PRISM 5700, 7000, 7700, and 7900_{HT} detection systems. The ABI detection systems contain a thermal cycler, fluorescence detection unit, and application-specific software for real-time detection of target sequences during each cycle of amplification using the methods of the present invention, and provides quantitative measurements of the detected target polynucleotides without additional purification or analysis following the amplification reaction.

Kits for Detecting Target Polynucleotides

The compositions and reagents employed in the methods described herein can be packaged into kits. In some embodiments, the kits can be used for detecting target sequences associated with infectious diseases, genetic disorders, or cellular disorders and, accordingly, for diagnosing such maladies.

Such diagnostic kits may include, for example, the labeled signal-quencher probe pairs and optional amplification primers. If the probes or primers are supplied unlabeled, the specific labeling reagents may also be included in the kit. The kit may also contain other suitably packaged reagents and materials needed for optional amplification, for example, buffers, dNTPs, and/or polymerizing means (e.g., reverse transcriptase and/or DNA polymerase), and for detection analysis, for example, enzymes and solid phase extractants, as well as instructions for conducting the assay.

In some embodiments, the kits can be used for determining the presence or absence of mutations or polymorphisms at multiple loci of one or more polynucleotides, comprising: 1) a first signal probe which is capable of hybridizing to a polynucleotide at a first target sequence and producing a first detectable fluorescent signal when hybridized thereto; 2) a first quencher probe capable of hybridizing in quenching proximity to the same target sequence as the first signal probe and quenching the signal of the first signal probe when hybridized in quenching proximity thereto, where the first quencher probe has a Tₘ below that of the first signal probe; 3) a second signal probe which is capable of hybridizing to the same or different polynucleotide at a second target sequence and producing a second detectable fluorescent signal when hybridized thereto, where the second signal probe has a Tₘ below that of the first quencher probe; and 4) an optional second quencher probe which is capable of hybridizing in quenching proximity to the same target sequence as the second signal probe and quenching the signal of the second signal probe when hybridized in quenching proximity thereto, where the optional second quencher probe has a Tₘ below that of the second signal probe.

Diagnostic Application of the Present Methods and Compositions

In some embodiments, the compositions and methods described herein can be used to detect target sequences associated with infectious diseases, genetic disorders, or cellular disorders and, thereby, diagnose such maladies. More particularly, the compositions and methods described herein can be used to detect mutations or sequence variations (e.g., genotyping), and polymorphisms, e.g., single nucleotide polymorphisms (SNPs) associated with such maladies.

The target sequences may be obtained from prokaryotes and eukaryotes, such as bacteria (including extremeophiles such as the archebacteria), fungi, insects, fish, shellfish, reptiles, and/or mammals. Suitable mammals include, but are not limited to, rodents (rats, mice, hamsters, guinea pigs, etc.), primates, farm animals (including sheep, goats, pigs, cows, horses, etc) and humans.

In some embodiments, the compositions and methods described herein can be used to detect SNPs associated with certain disease states. For example, some SNPs, particularly those in and around coding sequences, are likely to be the direct cause of therapeutically relevant phenotypic variants and/or disease predisposition. There are a number of well known polymorphisms that cause clinically important phenotypes; for example, the apoE2/3/4 variants are associated with different relative risk of Alzheimer's and other diseases (see Corder et al., (1993) Science 261: 828-9).

In some embodiments, the probes can be used in genetic diagnosis. For example, probes can be made using the techniques disclosed herein to detect target sequences such as the gene for nonpolyposis colon cancer, the BRCA1 breast cancer gene, P53, which is a gene associated with, a variety of cancers, the Apo E4 gene that indicates a greater risk of Alzheimer's disease, allowing for easy presymptomatic screening of patients, mutations in the cystic fibrosis gene, or any of the others well known in the art.

In some embodiments, the compositions and methods described herein can be used to detect bacterial sequences for diagnosis and/or genotyping. Bacterial sequences can be obtained from a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. Y. pestis, Pseudomonas, e.g. P. aeruginosa, P. putida; Chlamydia, e.g. C. trachomatis; Bordetella, e.g. B. pertussis; Treponema, e.g. T. palladium; and the like.

In some embodiments, the compositions and methods described herein can be used to detect viral sequences for diagnosis and/or genotyping. Viral sequences from any virus may be genotyped or identified using the compositions and methods described herein. Of particular interest, are RNA and DNA viruses that cause disease in humans. For example, RNA viruses belonging to Picornaviridae (e.g., Polioviruses 1-3, Hepatitis A), Caliciviridae, Astroviridae, Togaviridae, Flaviviridae (e.g., Hepatitis C Virus (HCV), Coronaviridae, Paramyxoviridae, Rhabdoviridae (e.g., Rabies virus), Filoviridae (e.g., Ebola virus), Orthomyxoviridae (e.g., Influenza viruses A and B), Bunyaviridae, Arenaviridae, Reoviridae, Birnaviridae and Retroviridae (e.g., human T-cell lymphoma viruses (HTLVs), human immunodeficiency virus (HIV)) can be detected using the compositions and methods described herein. Similarly, DNA viruses belonging to Hepadnaviridae (Hepatitis B), Circoviridae, Parvoviridae, Papillomaviridae (human papillomavirus), Polyomaviridae, Adenoviridae, Herpesviridae (e.g., human cytomegalovirus), Poxyiridae, and Iridoviridae can be detected using the compositions and methods described herein. See Fields “Virology” (2001), 4^{th} edition, Lippincott-Raven Publishers, Philadelphia, vols, 1 and 2; both of which are incorporated herein by reference in their entirety.

For example, in some embodiments, the compositions and methods described herein can be used to detect “virus specific sequences”. As used herein “virus specific sequences” refer to a target sequence having a genotype-specific sequence for a given virus. A “genotype-specific sequence” as used herein refers to a sequence that identifies a particular virus genotype and distinguishes that virus genotype from at least one other virus genotype, preferably from 3 to 5 other virus genotypes, and most preferably all other virus genotypes. Accordingly, in the methods described herein, a genotype-specific sequence probe discriminates between different viral genotypes by specifically binding to a sequence that identifies a particular viral genotype.

Methods of selecting virus genotype-specific sequences are known in the art. For example, HCV genotype-specific sequences can be selected by aligning the known HCV sequences and looking for variations between the sequences that distinguish one genotype from another. Further, HCV sequences can be isolated and sequenced and compared against known HCV sequences. In particular, DNA complements of the complete RNA genome of HCV have been cloned (see, e.g., Kato et al. Proc. Natl. Acad. Sci. USA 87:6547-6549 (1990); Choo et al., Proc. Natl. Acad. Sci. USA 88:2541-2455 (1991); Okamoto et al., J. Gen. Viral., 72:2697-2704 (1991); Okamoto et al. Virology 188:331-341 (1992)) and can be used to select HCV genotype-specific sequences. Methods of isolating and sequencing HCV isolates (e.g., from patient samples), as well as methods of selecting HCV genotype-specific sequences, are well known in the art. In addition, methods for aligning the known or isolated sequences and selecting HCV genotype-specific sequences based on differences or variations between known or isolated sequences are well known in the art (see, e.g., Lieven et al., Proc. Natl. Acad. Sci. USA 91:10134-10138 (1994); Leiven et al., Journal of Clinical Microbiology, 34(9):2259-2266 (1996)).

For example, sequences of interest can be aligned using the Lasergene software package from DNASTAR, Inc. Based on the results of the alignment, potential probes are identified and analyzed for the degree of secondary structure. The Tₘs of the potential probes are then calculated, and if necessary, the Tₘs are adjusted to obtain the desired Tₘs. The probes are then synthesized and the actual Tₘs measured. If the actual Tₘ differs significantly from the desired Tₘ, modified probes are designed and synthesized. A number of publications are available for predicting/calculating Tₘs. See for example, Santa Lucia et al., 1996, Biochemistry, 35:3555-3562, and Giesen et al., 1998, Nucleic Acids Research, 26:5004-5006.

Depending on the virus, several regions of the viral genome may be used to design probe sequences. For example, several regions of the HCV genome have been investigated with respect to genotyping and classification of HCV isolates. For example, 329 to 340 base pair non-structural (NS) 5B region, the core region, and the 5′ untranslated regions of the HCV genome are regions used in known methods of HCV genotyping (see, e.g., Stuyver et al, 1995, Virus Res. 38:137-157; Tokita et al., 1994, Proc. Natl. Acad. Sci. USA 91:11022-11026; Widell et al, 1994, J. Med. Virol. 44:272-279; Okamoto et al., 1993, J. Gen. Viral. 74:2385-2390; Smith et al., 1995, J. Gen. Virol. 76:1749-1761, all of which are incorporated herein by reference in their entireties.

In the methods described herein, multiple probes can be used to identify multiple viral genotypes in a multiplex assay, where the signal probes each bind to a different virus genotype-specific target sequence. For example, three different signal probes can be used in a single multiplex assay to detect the presence of three different virus genotype specific target sequences. Accordingly, the signal probes each contain a discriminating sequence that is complementary to a different virus genotype-specific target sequence. Similarly, three different quencher probes can be used in a single multiplex assay to detect the presence of three different virus genotype specific target sequences. Each quencher probe contains a discriminating sequence that is complementary to a different virus genotype-specific target sequence.

The following Examples are illustrative of the disclosed composition and methods, and are not intended to limit the scope of the compositions and methods described herein. Without departing from the spirit and scope of the compositions and methods described herein, various changes and modifications will be clear to one skilled in the art and can be made to adapt the compositions and methods described herein to various uses and conditions. Thus, other embodiments are encompassed.

The entire content of the specification for U.S. Ser. No. 60,448,440, filed Feb. 18, 2003, is hereby incorporated by reference in its entirety and for all purposes.

### EXAMPLES

### Example 1

### Target Sequence Discrimination Using Tₘ Multiplex Analysis

The ability of a Tₘ multiplex assay to unambiguously discriminate different target sequences was demonstrated with signal-quencher probe pairs specific for four different target sequences. The sequences of the targets, signal probes and quencher probes are provided in Table 1, below. The structure of the dye, “DYE 1” is shown below:

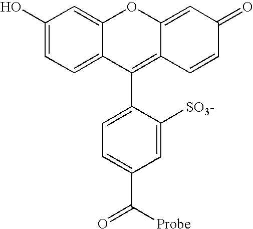

Signal probes were self-indicating linear PNA probes labeled at the amino terminus with DYE1 (signal label) and the carboxy terminus with a Dabcyl moiety (non-fluorescent quencher). The Dye1 and the Dabcyl were either linked directly to their respective termini, or spaced away using amino acid linkers, as indicated in Table 1.

TABLE 1

| | | |
|---|---|---|
| Name | Type | Sequence |

| | | |
|---|---|---|
| PNA 1 | PNA signal | Dye1-Glu-attgccaggacgacc-Lys-Lys(Dabcyl) |
| | probe | |
| PNA 2 | PNA signal | Dye1-Glu-attgccaggacgac-Lys-Lys(Dabcyl) |
| | probe | |
| PNA 3 | PNA signal | Dye1-attggggaca-Lys(Dabcyl) |
| | probe | |
| PNA 4 | PNA signal | Dye1-tggctaggga-Lys(Dabcyl) |
| | probe | |
| DNA-1 | DNA | GGACCCGGTCGTCCTGGCAATTCCGGTGTACTCACCGGT |
| | target | |
| DNA- | DNA | GCCTGTCCCCAATAGAATTGA |
| 18b | target | |
| DNA- | DNA | TCAGGATCCCTACCCATTTCCTGAA |
| 29a | target | |
| PNA 5 | PNA | Acetyl-tgagtacaccgg-Lys-Lys(Dabcyl) |
| | quencher | |
| | probe | |

In Table 1, Lys is the amino acid L-lysine and Glu is the amino acid L-glutamic acid.

Multiplex Tₘ, analyses was carried out on an ABI Prism 7700 Sequence detector. Each assay included one high Tₘ PNA signal probe (PNA 1 or PNA 2), its corresponding target DNA, one low Tₘ PNA signal probe (PNA 3 or PNA 4) and its corresponding target DNA. Each combination of high and low Tₘ probes was assayed twice: once with, and once without, PNA 5, a PNA quencher probe. The quencher probe hybridizes to the target sequence at a position one base downstream from the position where the PNA 1 and PNA 2 probes hybridize. The sample volume for each assay was 50 μL, and contained 5 μL 10× TaqMan Buffer A (Applied Biosystems, Foster City Calif.) and 1 μL each probe, target and quencher present. Final concentrations of the signal probes and targets were 0.2 μM unless otherwise noted. Final concentration of quencher probes was 0.4 μM.

For the assay, samples were heated rapidly to 95° C., then fluorescent data was collected from 90° C. to 30° C. at a rate of 0.33° C./min. All assays were run in triplicate. The fluorescent signal of Dye1 was measured using the Sequence Detector software. The derivative of these signals (derivative profile) was calculated using Dissociation Curves software packaged with the fluorescence spectrophotometer instrument.

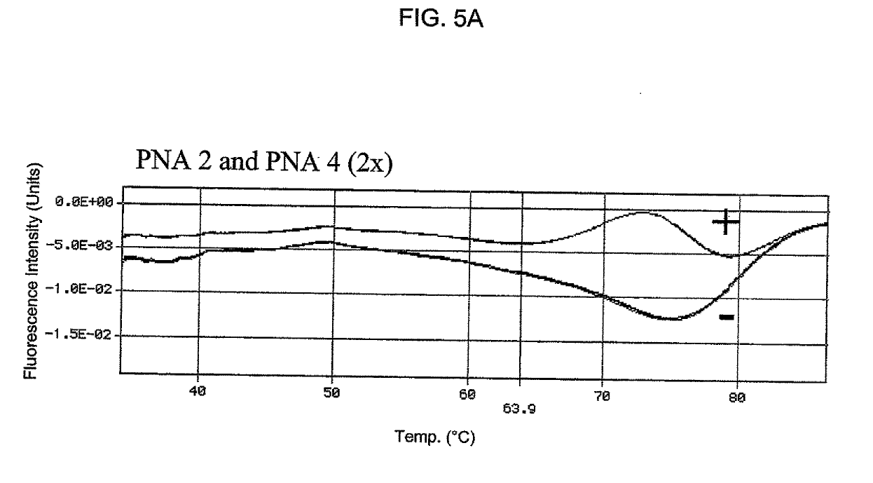

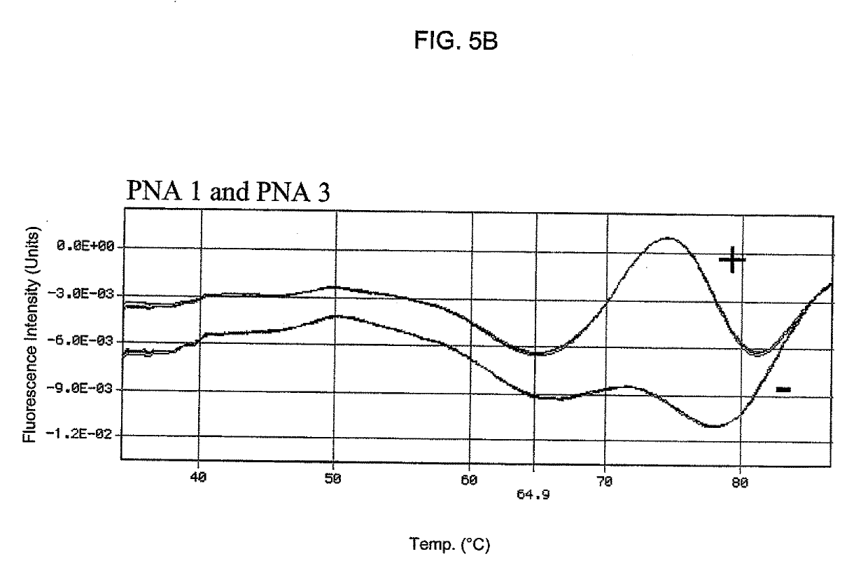

The derivative profile of the fluorescent signal for an assay performed with signal probes PNA 2 and PNA 4 is provided in FIG. 5A. The derivative profile for an assay performed with signal probes PNA 1 and PNA 3 is provided in FIG. 5B. In each figure, the profile labeled with a plus (“+”) is from the assay performed in the presence of the quencher probe. The profile labeled with a minus (“−”) is from the assay performed in the absence of the quencher probe. As noted, in FIG. 5A, the PNA signal probe PNA 4 was used at a 2× concentration (0.4 μM).

Referring to FIG. 5A, looking at the “+” profile and following the temperature downward (from right to left), a valley followed by a peak, followed by a second valley is observed. Looking at the “−” profile in the same manner, only a valley and no distinct peaks are observed. The only difference between the two assays is the presence of the quencher probe “turning off” the signal from the first, high Tₘ signal probe (PNA 2 signal probe). The Tₘ of the PNA 2 signal probe is approx. 77° C., whereas the Tₘ of the PNA 4 signal probe is approx. 66° C. (yielding a difference of approx. 11° C.). The Tₘ of the quencher probe (PNA 5) is approx. 72° C. When the temperature is cooled over the 90-30° C. range, the order in which the probes hybridize is PNA 2>PNA 5>PNA 4. The observed valley-curve-valley of the “+” profile represents the distinct probe-quencher hybridizations. In contrast, in the “−” curve, the observed valley is the composite of the fluorescent signals of the PNA 2 and PNA 4 signal probes as they hybridize at their respective Tₘs. The resultant signal, occurring as peak and valleys at or a around a particular Tₘ is diagnostic for this particular combination of probes and targets.

The profiles of FIG. 5B are similar to those of FIG. 5A. In this experiment, both signal probes were present at 0.2 μM. Again, a very distinct valley-peak-valley signature is evident in the profile obtained in the presence of the quencher probe (“+” profile). The profile obtained in the absence of the quencher probe (“−” profile) also displays a valley-peak-valley signature, but it is less distinct. In this example, the Tₘ difference between the two signal probes is greater than that of the previous example (Tₘ^{PNA 1}≈79° C.; Tₘ^{PNA 3}≈64° C., yielding a different of approx. 15° C.).

Although the above example demonstrates the ability of a Tₘ multiplex assay to unambiguously discriminate closely related sequences, this assay can also be used to discriminate polymorphisms associated with certain disease states. There are a number of well known polymorphisms that cause clinically important phenotypes; for example, the apoE2/3/4 variants are associated with different relative risk of Alzheimer's and other diseases (see Corder et al., Science (1993) 261: 828-9), sickle cell anemia, phenylketonuria, hemophilia, cystic fibrosis, and various cancers have been associated with one or more genetic mutation(s). Probe pairs may be designed to detect single base mutations associated with these diseases.

### Example 2

### Tₘ Multiplex Analysis Effectively Increases the Specificity of Signal Probes

This example demonstrates the ability of Tₘ multiplex assays to effectively increase the specificity of signal probes, permitting discrimination between extremely closely related target sequences.

For this example, an experiment similar to that described in Example 1 was performed using a target DNA including a mismatch to the PNA 1 probe. In this experiment, signal from the PNA 1 probe was not recorded in the “+” derivative profile, because the Tₘ of the signal probe-target sequence hybrid was below the Tₘ of the quencher probe-target hybrid. Since the quencher probe hybridized first, signal from the signal probe was quenched and not observed (only very weak signal detected). In the absence of the quencher probe, signal was observed at the lower temperature. Thus, by quenching signal from mismatched hybrids, the use of the quencher probe effectively increased the specificity of the signal probe.

### Example 3

### Genotype Discrimination Using Tₘ Multiplex Analysis

The ability of a Tₘ multiplex assay to unambiguously discriminate closely related HCV genotype sequences was demonstrated with signal-quencher probe pairs specific for particular HCV genotype sequences and synthetic HCV-specific DNA targets. The sequences of the probes and target sequences are provided in Table 2, below. Dye 1 is the same dye as used in Example 1. The underlined nucleotides depict the sequence to which the signal probes hybridize.

TABLE 2

| | | | | Probe | Target |
|---|---|---|---|---|---|
| Probe | Sequence | Target Sequence | Tm | (μM) | (μM) |
| HCV | Dye1-Glu-cggaattgccaggacg-Lys-Lys(Dabcyl) | GGACCCGGTCGTCCTGGCAATTCCGGTGTACTCACCGGT | 82 | 0.2 | 0.2 |
| 1 | | | | | |
| HCV | cggtgagtacac-Lys-Lys(Dabcyl) | GGACCCGGTCGTCCTGGCAATTCCGGTGTACTCACCGGT | 72 | 0.4 | — |
| 2 | | | | | |
| HCV | Dye1-Glu-ggccttgtggtac-Lys-Lys(Dabcyl) | GCAGTACCACAAGGCCTTTCGCGACCCAACA | 67 | 0.2 | 0.2 |
| 3 | | | | | |
| UnQ | Ac-ggt-cg-Gly-Gly-cga-aa-lys-Lys(Dabcyl) | GCAGTACCACAAGGCCTTTCGCGACCCAACA | 60 | 0.4 | — |
| SNP | Dye1-ggagaaactg-Lys(Dabcyl) | CAGCATGTACAGTTTCTCCAATACC | 60 | 0.2 | 0.2 |

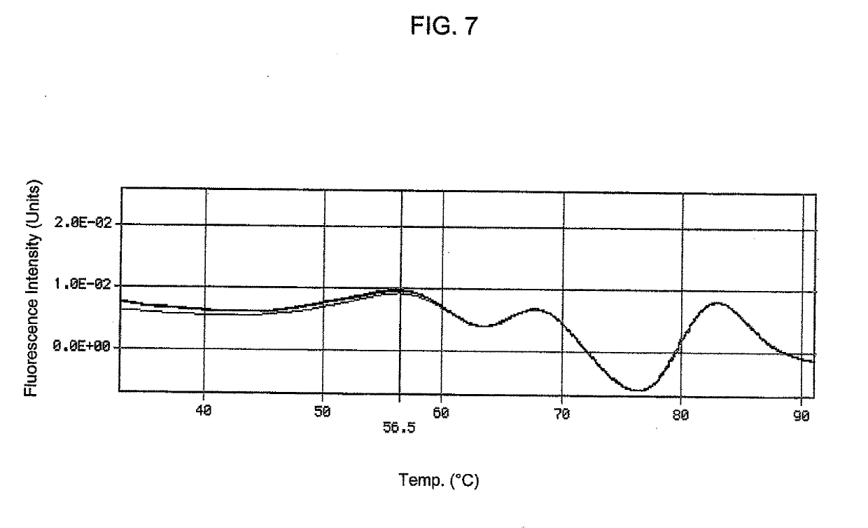

Multiplex analyses were carried out as described in Example 1. FIG. 7 shows the first derivative of the fluorescence from a hybridization experiment involving the 5 PNA and 3 (synthetic) DNA molecules shown in Table 2. Increases in fluorescent signal show up as peaks in the first derivative as opposed to valleys in the cooling curves. The temperature ranges used are shown on the X axis in FIG. 7. Samples were run in triplicate. The data was collected by heating from 30° C. to 95° C. over 19:59 minutes following a 19:59 minute cooling step from 95° C. to 30° C. (cooling data not shown).

Five hybridization events are apparent in FIG. 7. These events are indicated by the three peaks observed at approximately 56, 68 and 83° C. and the two valleys observed at approximately 63 and 76° C. These results indicate that closely related sequences can be readily discriminated using the methods of the present invention.

All references cited herein are expressly incorporated by reference in their entirety and for all purposes.

1. A method of analyzing a polynucleotide sample for one or more target sequences, comprising the steps of:
contacting a polynucleotide sample suspected of comprising one or more target sequences with:
(i) a first signal probe which hybridizes to at least a portion of a first target sequence and produces a first detectable signal when hybridized thereto;
(ii) a first quencher probe which hybridizes to the first target sequence in quenching proximity to the first signal probe and decreases the signal of the first signal probe when hybridized in quenching proximity thereto, said first quencher probe having a Tₘ below that of the first signal probe;
(iii) at least a second signal probe which hybridizes to at least a portion of a second target sequence and produces a second detectable signal when hybridized thereto; and
(iv) an optional second quencher probe which hybridizes to the second target sequence in quenching proximity to the second signal probe and decreases the signal of the second signal probe when hybridized in quenching proximity thereto, said optional second quencher probe having a Tₘ below that of the second signal probe;
directly detecting the detectable signals of the signal probes as a function of temperature; and
determining therefrom the presence or absence of one or more target sequences in said polynucleotide sample.

2. The method of claim 1 in which the first and second detectable signals are fluorescent signals.

3. The method of claim 2 in which the first and second fluorescent signals are spectrally resolvable.

4. The method of claim 1 in which the Tₘ of the first signal probe is higher than the Tₘ of the second signal probe.

5. The method of claim 1 in which the Tₘ of the first quencher probe is in the range of about 5 to 10° C. lower than that of the first signal probe and the Tₘ of the optional second quencher probe is in the range of about 5 to 10° C. lower than that of the second signal probe.

6. The method of claim 2 in which the first and second fluorescent signals are not spectrally resolvable, and the second signal probe has a lower Tₘ than the first quencher probe.

7. The method of claim 6 in which the Tₘ of the first quencher probe is in the range of about 5 to 10° C. lower than that of the first signal probe and the Tₘ of the optional second quencher probe is in the range of about 5 to 10° C. lower than that of the second signal probe.

8. The method of claim 6 in which the Tₘ of the second signal probe is in the range of about 7 to 15° C. lower than that of the first signal probe.

9. The method of claim 6 in which the first and second fluorescent signals are the same.

10. The method of claim 1 in which the optional second quencher probe is present.

11. The method of claim 1 in which the first and second signal probes are self-indicating signal probes.

12. The method of claim 11 in which the self-indicating probes are hairpin probes.

13. The method of claim 12 in which the first signal, first quencher, second signal and optional second quencher probes are resistant to degradation by nucleases.

14. The method of claim 12 in which the first signal, first quencher, second signal and optional second quencher probes are each, independently of one another, selected from the group consisting of a DNA nucleobase oligomer, an RNA nucleobase oligomer and a PNA nucleobase oligomer.

15. The method of claim 14 in which the first signal, first quencher, second signal and optional second quencher probes are all DNA, RNA or PNA nucleobase oligomers.

16. The method of claim 11 in which the self-indicating probes are linear self-indicating probes.

17. The method of claim 16 in which the first signal, first quencher, second signal and optional second quencher probes are resistant to degradation by nucleases.

18. The method of claim 16 in which the first signal, first quencher, second signal and optional second quencher probes are each, independently of one another, selected from the group consisting of DNA, RNA and PNA nucleobase oligomers.

19. The method of claim 18 in which the first signal, first quencher, second signal and optional second quencher probes are all DNA, RNA or PNA nucleobase oligomers.

20. The method of claim 18 in which the first signal, first quencher, second signal and optional second quencher probes are all PNA nucleobase oligomers.

21. The method of claim 11 in which each self-indicating probe includes a label which is capable of distinguishing hybridized from unhybridized signal probe.

22. The method of claim 21 in which the label is a fluorescent intercalating dye.

23. The method of claim 22 in which the fluorescent intercalating dye is selected from the group consisting of acridine orange, ethidium bromide, propidium iodide, hexium iodide, ethidium bromide homodimer, 3,3′-diethylthiadicarbocyanine iodide, SYBR® Green I and SYBR® Green II, 7-aminoactinomycin D, and actinomycin D.

24. The method of claim 21 in which the label is a fluorescent minor-groove-binding dye.

25. The method of claim 24 in which the fluorescent minor-groove-binding dye is selected from the group consisting of bisbenzimide dyes.

26. The method of claim 1 in which the detectable signals are detected as a function of decreasing temperature from a temperature above the Tₘ of the first signal probe to a temperature below the Tₘ of the optional second quencher probe.

27. The method of claim 26 in which the detectable signals are detected at temperatures approximately equal to the Tₘs of the signal and quencher probes.

28. The method of claim 26 in which the detectable signals are detected at temperatures approximately halfway between the Tₘs of the signal and quencher probes.

29. The method of claim 26 in which the temperature is decreased at a rate in the range of about 0.01° C./minute to about 5° C./minute.

30. The method of claim 26 in which the detectable signals are detected continuously at a rate in the range of about every 100 to 10,000 msec as a function of temperature.

31. The method of claim 1 in which the detectable signals are detected as a function of increasing temperature from a temperature below the Tₘ of the optional second quencher probe to a temperature above the Tₘ of the first signal probe.

32. The method of claim 31 in which the detectable signals are detected at temperatures approximately equal to the Tₘs of the signal and quencher probes.

33. The method of claim 31 in which the detectable signals are detected at temperatures halfway between the Tₘs of the signal and quencher probes.

34. The method of claim 31 in which the temperature is increased at a rate in the range of about 0.01° C./minute to about 5° C./minute.

35. The method of claim 31 in which the detectable signals are detected continuously at a rate in the range of about every 100 to 10,000 msec as a function of temperature.

36. The method of claim 1 in which the detectable signals are detected as a function of temperature by determining the Tₘs of the first and second signal probes.

37. The method of claim 1 in which the polynucleotide sample is selected from the group consisting of genomic DNA, cDNA, RNA, mRNA, rRNA and an amplification product.

38. The method of claim 37 in which the polynucleotide sample is single-stranded.

39. The method of claim 1 in which the polynucleotide sample comprises two or more different polynucleotides.

40. The method of claim 1 in which the target sequences are present on two or more polynucleotides.

41. The method of claim 1 in which the target sequence is present on the same polynucleotide strand.

42. The method of claim 1 in which the target sequence is present on two different polynucleotide strands.

43. A method of analyzing a polynucleotide sample for one or more target sequences, comprising the steps of:
contacting a polynucleotide sample with:
(1) a first set of m signal-quencher probe pairs, each of which comprises
(i) a signal probe which hybridizes to a portion of a target sequence and produces a first detectable signal when hybridized thereto and
(ii) a corresponding quencher probe which hybridizes in quenching proximity to the signal probe and quenching decreases its detectable signal when hybridized in quenching proximity thereto, wherein the first signal probe has the highest Tₘ and the Tₘ of each quencher probe is lower than the Tₘ of its corresponding signal probe and the Tₘ of each signal probe is lower than the Tₘ of the quencher probe of the preceding signal-quencher probe pair, and further wherein the quencher probe of the signal-quencher probe pair of the first set having the lowest Tₘ is optional; and
(2) a second set of n signal-quencher probe pairs, each of which comprises
(i) a signal probe which hybridizes to a portion of a target sequence and produces a second detectable signal distinguishable from the first detectable signal when hybridized thereto and
(ii) a corresponding quencher probe which hybridizes in quenching proximity to the signal probe and decreases its detectable signal when hybridized in quenching proximity thereto, wherein the Tₘ of each quencher probe is lower than the Tₘ of its corresponding signal probe and the Tₘ of each signal probe is lower than the Tₘ of the quencher probe of the preceding signal-quencher probe pair, and further wherein the quencher probe of the signal-quencher probe pair of the second set having the lowest Tₘ is optional;
directly detecting the first and second detectable signals as a function of temperature; and
determining the presence or absence of one or more target sequences in said polynucleotide sample.

44. A method of genotyping an organism, comprising the steps of:
contacting a polynucleotide sample from the organism, or an amplification product thereof, with a first plurality of signal-quencher probe pairs, each of which hybridizes, in quenching proximity, to a different genotype-specific sequence and produces a resolvable, temperature-dependent hybridization profile;
obtaining temperature-dependent hybridization profiles for the signal-quencher probe pairs, which comprises plotting the signal intensity during the decrease of the signals of the signal probes when the temperature is below the Tₘ of their corresponding quencher probes and plotting the signal intensity during the increase of the signals of the signal probes when the temperature is below the Tₘ of their corresponding quencher probes; and
determining therefrom the genotype of the organism.

45. A method of genotyping a virus, comprising the steps of:
contacting a polynucleotide sample from a virus, or an amplification product thereof, with a first plurality of signal-quencher probe pairs, each of which hybridizes, in quenching proximity, to a different virus genotype-specific sequence and produces a resolvable, temperature-dependent hybridization profile;
obtaining temperature-dependent hybridization profiles for the signal-quencher probe pairs, which comprises plotting the signal intensity during the decrease of the signals of the signal probes when the temperature is below the Tₘ of their corresponding quencher probes and plotting the signal intensity during the increase of the signals of the signal probes when the temperature is below the Tₘ of their corresponding quencher probes; and
determining therefrom the genotype of the virus.

46. A method of analyzing a sample for the presence of a polynucleotide sequence of interest, comprising the steps of:
contacting a polynucleotide from the sample, or an amplification product thereof, with a first plurality of signal-quencher probe pairs, wherein each said signal-quencher probe pair hybridizes, in quenching proximity, to a different known target sequence and produces a resolvable, temperature-dependent hybridization profile;
obtaining temperature-dependent, hybridization profiles for the signal-quencher probe pairs, which comprises plotting the signal intensity during the decrease of the signals of the signal probes when the temperature is below the Tₘ of their corresponding quencher probes and plotting the signal intensity during the increase of the signals of the signal probes when the temperature is below the Tₘ of their corresponding quencher probes; and
determining the presence or absence of one or more different target sequences.

47. A multiplex method of genotyping a polynucleotide of an organism, comprising the steps of:
amplifying the polynucleotide in the presence of amplification primers suitable for producing a plurality of genotype-specific amplicons and a plurality of signal-quencher probe pairs, wherein each said signal-quencher probe pair hybridizes, in quenching proximity, to a different genotype-specific amplicon and produces a resolvable, temperature-dependent, hybridization profile;
obtaining temperature-dependent, hybridization profiles for the signal-quencher probe pairs, which comprises plotting the signal intensity during the decrease of the signals of the signal probes when the temperature is below the Tₘ of their corresponding quencher probes and plotting the signal intensity during the increase of the signals of the signal probes when the temperature is below the Tₘ of their corresponding quencher probes; and
determining therefrom the genotype of the organism.

48. A multiplex method of diagnosing a patient for a malady of interest, comprising the steps of:
incubating a polynucleotide sample derived from the patient in the presence of a plurality of signal-quencher probe pairs, wherein each said signal-quencher probe pair hybridizes, in quenching proximity, to a different target sequence indicative of a particular malady of interest and produces a resolvable, temperature-dependent, hybridization profile when hybridized thereto;
obtaining temperature-dependent, hybridization profiles for the signal-quencher probe pairs, which comprises plotting the signal intensity during the decrease of the signals of the signal probes when the temperature is below the Tₘ of their corresponding quencher probes and plotting the signal intensity during the increase of the signals of the signal probes when the temperature is below the Tₘ of their corresponding quencher probes; and
determining therefrom whether the patient has the malady of interest.

49. A multiplex method of diagnosing a patient for a malady of interest, comprising the steps of:
amplifying a polynucleotide sample derived from the patient in the presence of amplification primers suitable for producing a plurality of different amplicons, each of which correlates to a different malady of interest, and a plurality of signal-quencher probe pairs, wherein each said signal-quencher probe pair hybridizes, in quenching proximity, to a different amplicon and produces a resolvable, temperature-dependent, hybridization profile;
obtaining temperature-dependent, hybridization profiles for the signal-quencher probe pairs, which comprises plotting the signal intensity during the decrease of the signals of the signal probes when the temperature is below the Tₘ of their corresponding quencher probes and plotting the signal intensity during the increase of the signals of the signal probes when the temperature is below the Tₘ of their corresponding quencher probes; and
determining therefrom whether the patient has the malady of interest.

50-51. (canceled)

52. The method of claim 1, wherein each of the first quencher probe and optionally second quencher probe is non-fluorescent.

53. The method of any one of claims 43-49, wherein the quencher probes are non-fluorescent.

54. The method of claim 1, wherein the detectable signal from the signal probe is measured as a function of decreasing temperature or as a function of increasing temperature.
